(19) United States
(12) Patent Application Publication
(10) Pub. No.: US 2004/0132108 A1
(43) Pub. Date: Jul. 8, 2004

(54) Screening methods and agents

(76) Inventors: Theodore Robert Hupp, Dundee (GB); David Dornan, Dundee (GB)

Correspondence Address: MYERS BIGEL SIBLEY & SAJOVEC, PO BOX 37428, RALEIGH, NC, 27627, US

(21) Appl. No.: 10/467,758

(22) PCT Filed: Feb. 13, 2002
(86) PCT No.: PCT/GB02/00640

(30) Foreign Application Priority Data
Feb. 13, 2001 (GB) 0103508.8

(51) Int. Cl. G01N 33/53; G01N 33/567

(52) U.S. Cl. 435/7.2

(57) ABSTRACT

The present invention relates to a method for screening for agents which modulate the binding of p53 to p300, and agents identified using such an assay. Such agents include peptide mimetics of regions of p53 and/or p300 which have been identified as contact region for the other protein. The agents of the present invention are candidates for use in the treatment of, for example cancer, or ischemia.

### FIELD OF THE INVENTION

The present invention relates to an method for screening for agents which modulate the binding of p53 to p300, and agents identified using such an assay. Such agents are candidates for use in the treatment of, for example, cancer, or ischemia.

### BACKGROUND OF THE INVENTION

The link between the role of p53 as a tumour suppressor and its activity as a transcription factor has been well documented (Oren, 1999). More recent efforts have concentrated on the post-translational events upstream of p53 that activate its tumour suppressor function. Such studies have highlighted the fact that two key proteins play a role in regulating p53 function. The MDM2 protein forms a component of a negative regulatory pathway that facilitates ubiquitin-dependent degradation of p53 through the proteosome (Lohrum and Vousden, 2000). The p300 transcriptional adaptor protein forms a component of a positive regulatory pathway that facilitates the induction of p53-dependent gene expression (Goodman and Smolik, 2000).

Post-translational modification of the N-terminal BOX-I domain of p53 via kinase phosphorylation is known to occur and is thought to influence the specific activity of p53 as a tumour suppressor. The role of phosphorylation in modulating p53 function becomes apparent during senescence, quiescence, or after exposure to DNA damaging agents, where steady-state phosphorylation of p53 increases at Ser¹⁵ (Webley et al., 2000). Phosphorylation at Ser¹⁵ increases binding to CBP (Lambert et al., 1998) and p300 (Dumaz and Meek, 1999), and simultaneously decreases binding to MDM2 (Shieh et al., 1997), highlighting the physiological importance of this phosphorylation event in yielding a transcriptionally competent form of p53. One other newly identified phospho-acceptor site at Thr¹⁸ in the BOX-I domain is modified in human breast cancers (Craig et al., 1999b), induced during senescence (Webley et al., 2000) or transiently following ionising radiation (Sakaguchi et al., 2000). The second newly-identified phospho-acceptor site at Ser²⁰ is modified constitutively in normal human fibroblasts and oxidant stresses including radiolabeling with ³²P-orthophosphate can result in de-phosphorylation at this site (Craig et al., 1999a). In addition, an intact Ser²⁰ residue is required for effective p53 activity (Unger et al., 1999) and the ionising irradiation-induced form of p53 protein is phosphorylated at Ser²⁰ by a Chk2-dependent pathway (Shieh et al., 2000). However, conflicting evidence (Ashcroft et al., 1999) suggests that Thr¹⁸ and/or Ser²⁰ phosphorylation events have no effect on p53 activity. Moreover, the overlap of the MDM2 binding site and the p300-binding site within the BOX-I domain of p53 complicates an understanding of the role of the Thr¹⁸ and BOX-I domain phosphorylation sites on p53 function.

The transcriptional co-activator p300 plays an essential role in the tumour suppressor activity of p53. The specific interaction of p300 in the p53-dependent transactivation pathway became apparent when it was demonstrated that ectopically expressed p300 stimulated p53-dependent gene expression and that adenoviral E1A protein inhibited p53-dependent transcription by virtue of binding to p300 (Avantaggiati, Ogryzko et al. 1997; Gu, Shi et al. 1997; Lill, Grossman et al. 1997). Although this initial p300-p53 interaction was mapped to the N-terminal BOX-1 domain of p53, an additional role for p300 in the control of p53 activity came from the observation that acetylation of p53 at its C-terminal negative regulatory domain by p300 activated the specific DNA binding function of p53 to short oligonucleotides containing the consensus binding site for p53 (Gu and Roeder 1997). Together, these data suggest the existence of a phosphorylation-acetylation cascade that targets p53 in response to genotoxic stress (Lambert, Kashanchi et al. 1998; Sakaguchi, Herrera et al. 1998) However, more recently it was demonstrated that acetylation cannot activate the specific DNA binding function of p53 on long DNA fragments containing the p53 consensus binding site (Espinosa and Emerson 2001), as has been observed with phosphorylation by CDK2 or PKC (Blaydes, Luciani et al. 2001), suggesting that acetylation may have roles other than regulating DNA binding by p53. Further, using an in vitro transcription system containing a chromatin-assembled promoter, it was shown that the C-terminal domain is essential for in vitro transcription (Espinosa and Emerson 2001). Thus, it appears that although the C-terminal domain of p53 negatively regulates its activity with respect to sequence-specific DNA binding, it acts as a positive regulatory domain with respect to p300-dependent transcription but the actual role of p53 acetylation by p300 remains unclear (Prives and Manley 2001).

### SUMMARY OF THE INVENTION

The present invention is therefore based in part on the results of studies into the role of Thr¹⁸ and Ser²⁰ phosphorylation sites in regulating p53 function, in particular binding to p300.

It is amongst the objects of the present invention to provide a method of screening candidate agents for any modulatory effect on a p53/p300 complex.

In a first aspect the present invention provides a method for identifying a substance capable of modulating an interaction between (i) a p53 polypeptide or a homologue thereof, or a derivative thereof, and (ii) a p300 polypeptide, or a homologue thereof, or a derivative thereof, which method comprises:

a) providing a p53 polypeptide or a homologue, or a derivative thereof, as a first component;

b) providing a p300 polypeptide or a homologue, or a derivative thereof, as a second component;

c) contacting the two components with a test substance under conditions that would permit the two components to bind in the absence of said test substance; and

d) determining whether said substance modulates the interaction between the first and second components.

The method may further comprise

e) administering a substance which has been determined to disrupt the interaction between the first and second components to an animal cell; and

f) determining the effect of the substance on the cell.

It is understood that the term “modulation” refers to both positive and negative modulation. “Positive modulation”, as used herein refers to an increase in the binding of p53 polypeptide or a homologue thereof, or a derivative thereof to p300 or a homologue thereof, or a derivative thereof relative to the level of binding and/or activity as a result of the binding in the absence of the substance. “Negative modulation” as used herein refers to a decrease in the binding of p53 polypeptide or a homologue thereof, or a derivative thereof to p300 or a homologue thereof, or a derivative thereof relative to the level of binding and/or activity as a result of the binding in the absence of the substance.

The invention further provides a substance capable of modulating an interaction between (i) a p53 polypeptide or a homologue thereof, or a derivative thereof, and (ii) p300 or a homologue thereof, or a derivative thereof, for use in treating the human or animal body by therapy or for use in diagnosis, whether or not practised on the human or animal body. Such a substance may thus be used in the prevention or treatment of for example cancer, or ischemia.

The invention therefore further provides a substance capable of modulating an interaction between (i) a p53 polypeptide or a homologue thereof, or a derivative thereof, and (ii) p300 or homologues thereof, or derivatives thereof, for use in regulating the cell cycle of a mammalian cell. The substance may be used for modulating growth arrest and/or cell death. In that event, the mammalian cell may for example be a tumour cell.

The invention also provides a method of regulating the cell cycle in a mammalian cell, which method comprises administering to said cell a substance capable of modulating an interaction between (i) a p53 polypeptide or a homologue thereof, or a derivative thereof, and (ii) p300 or a homologue thereof, or a derivative thereof.

Examples of suitable substances include peptide mimetics based on the BOX-I domain of p53 particularly peptides comprising phosphorylated—Ser²⁰ of the BOX-I domain, or a mutated version designed to mimic phosphorylated-Ser²⁰ (eg. when Ser²⁰ is replaced by a aspartate). Alternative peptide mimetics may comprise polyproline regions designed to mimic a polyproline binding region on p300 that binds to a polyproline domain on p53.

### DETAILED DESCRIPTION OF THE INVENTION

Polypeptide Components

The first component comprises a p53 polypeptide or a homologue thereof or a derivative of p53 or of a p53 homologue. p53 is a well-known tumour suppressor protein described for example in Oren, M (1999). Homologues of p53 include p63 and p73. Derivatives of p53 include fragments of p53 which comprise at least a region having substantial homology to the BOX-I domain of p53 (May and May, 1999). The fragments may be up to 40, 50, 60 or 100 amino acid residues long. The minimum fragment length may be 5, 10, 20 or 30 amino acid residues. Herein, substantial homology for fragments of p53 is regarded as a sequence which has at least 70%, e.g. 80%, 90% or 95%, amino acid homology (identity) over 10, preferably 15, more preferably 20 amino acids with the BOX-I domain of p53. p53 fragments may be phosphorylated typically at phospho-acceptor sites of the BOX-I domain, eg. Ser¹⁵, Thr¹⁸ and/or Ser²⁰ (numbering according to position of amino acid upon p53 protein).

Alternatively phospho-peptide mimetics may be used in which phospho acceptor sites, such as Ser¹⁵, Thr¹⁸ and/or Ser²⁰ are substituted with aspartate residues or glutamate.

Alternative peptide mimetics include peptides comprising the motif PXnP where n is 1 to 3 and X is any amino acid.

Derivatives further include variants of p53 and its homologues or derivatives, including naturally occurring allelic variants and synthetic variants which are substantially homologous to said p53 and its homologues.

The second component is selected from p300 or homologues thereof, and their derivatives (Goodman & Smolik, 2000). Derivatives of p300 include fragments, preferably comprising at least 30 amino acids, more preferably at least 50 amino acids, which are capable of binding to p53. Such fragments include fragments containing the N-terminus of p300. Derivatives further include variants of p300, its homologues or derivatives, including naturally occurring allelic variants and synthetic variants which are substantially homologous to said p300. In this context, substantial homology is regarded as a sequence which has at lest 70%, eg. 80% or 90% amino acid homology (identity) over 30, preferably 50, more preferably 60 amino acids with p300.

Preferred fragments include phospho-serine²⁰ binding domains of p300 found in the C and N terminal regions of p300 and sites for polyproline contact found on p300.

It will be understood that for the particular polypeptides embraced herein, natural variations such as may occur due to polymorphisms, can exist between individuals or between members of the family. These variations may be demonstrated by (an) amino acid difference (s) in the overall sequence or by deletions, substitutions, insertions, inversions or additions of (an) amino acid(s) in said sequence. All such derivatives showing the recognised modulatory activity are included within the scope of the invention. For example, for the purpose of the present invention conservative replacements may be made between amino acids within the following groups:

(I) Alanine, serine, threonine;

(II) Glutamic acid and aspartic acid;

(III) Arginine and leucine;

(IV) Asparagine and glutamine;

(V) Isoleucine, leucine and valine;

(VI) Phenylalanine, tyrosine and tryptophan.

Derivatives may be in the form of a fusion protein wherein p53 and/or p300, a homologue or derivative thereof is fused, using standard cloning techniques, to another polypeptide which may, for example, comprise a DNA-binding domain, a transcriptional activation domain or a ligand suitable for affinity purification (for example glutathione-S-transferase or six consecutive histidine residues).

The first and second components used in the assays may be obtained from mammalian extracts, produced recombinantly from, for example, bacteria, yeast or higher eukaryotic cells including mammalian cell lines and insect cell lines, or synthesised de novo using commercially available synthesisers. Preferably, the first and second components used in the assays are recombinant.

Candidate Substances

A substance which modulates an interaction between the first component and the second component may do so in several ways. It may directly modulate the binding of the two components by, for example, binding to one component and masking or altering the site of interaction with the other component. Candidate substances of this type may conveniently be screened by in vitro binding assays as, for example, described below. Examples of candidate substances include non-functional homologues of the first or second components as well as antibodies which recognise the first or second components.

A substance which can bind directly to the first or second component may also inhibit an interaction between the first component and the second component by altering their subcellular localisation thus preventing the two components from coming into contact within the cell. This can be tested in vivo using, for example the in vivo assays described below. The term “in vivo” is intended to encompass experiments with cells in culture as well as experiments with intact multicellular organisms.

Suitable candidate substances include peptides, especially of from about 5 to 20 amino acids in size, based on the sequence of the BOX-I domain of p53, or variants of such peptides in which one or more residues have been substituted (for example Ser¹⁵, Thr¹⁸ and/or Ser²⁰ substituted by aspartate or glutamate), as described herein. Such peptides may also be fused to other proteins/peptide such as Green Fluorescent Protein (GFP), which may serve as a marker.

Particularly preferred peptides comprise the sequence S^{PO3}XXWKLL where S^{PO3} represents a phosphorylated serine and X is any amino acid, which is the consensus BOX-I p300-binding motif on p53. Naturally a region or regions on p300 to which such peptides bond are also preferred. Two such regions have been identified by the present inventors and are herein after referred to as POD1 and POD2, having the sequences as follows:

| | |
|---|---|
| POD1: | CASSRQIISHWKNCTRHDCPVCLPLKNAGDKRNQQPILTGAPVGLGNPSSLGVGQQS |
| | APNLSTVSQIDPSSIERAYAALGLPYQVNQMPTQPQVQAKNQQNQQPGQSPQGMRP |
| | MSNMSASPMGVNGGVGVQTPSLLSDSMLHSAINSQNPMMSENASVPSLGPMPTAAQ |
| | PSTTG |
| POD2: | AAGQVTPPTPPQTAQPPLPGPPPTAVEMAMQIQRAAETQRQMAHVQIFQRPIQHQMP |
| | PMTPMAPMGMNPPPMTRGPSGHLEPGMGPTGMQQQPPWSQGGLPQPQQLQSGMP |
| | RPAMMSVAQHGQPLNMAPQPGLGQVGISPLKPGTVSQQALQNLLRTLRSPSSPLQQQ |
| | QVLSILHANPQLLAAFIKQRAAKYANSNPQPIPGQPGMPQGQPGLQPPTMPGQQGVHS |
| | NPAMQNMNPMQAGVQRAGLPQQQPQQQLQPPMGGMSPQAQQMNMNHNTMPSQFR |
| | DILRRQQMMQQQQQQGAGPGIGPGMANHNQFQQPQGVGYPPQPQQRMQHHMQQM |
| | QQGNMGQIGQLPQALGAEAGASLQAYQQRLLQQQMGSPVQPNPMSPQQHMLPNQ |
| | AQSPHLQGQQIPNSLSNQVRSPQPVPSPRPQSQPPHSSPSPRMQPQPSPHHVSPQT |
| | SSPHPGLVAAQANPMEQGHFASPDQNSMLSQLASNPGMANLHGASATDLGLSTDNS |
| | DLNSNLSQSTLDIH |

Further preferred peptides may be based on peptides comprising regions of polyprolines with the motif PXP, PXXP or PXXXP. One such region found on p53, comprises the sequence: PRMPEMPPVAPAPAAPTPAAPAPAPSWP as well as sites for polyproline contact found on p300. The present inventors have found two such polyproline contact sites on p300, hereinafter termed SPC1 and SPC2 found at the N and C terminal region, of p300 respectively. The sequences of SPC1 and SPC2 are as follows:

| | |
|---|---|
| SPC1: | PAMGMNTGTNAGMNPGMLAAGNGQGIMPNQVMNGSIGAGRGRQDMQYPNPGM |
| | GSAGNLLTEPLQQGSPQMGGQTGLRGPQPLKMGMMNNPNPYGSPYTQNPGQQI |
| | GASGLGLQIQTKTVLSNNLSPFAMDKKAVPGGGMPNMGQQPAPQVQQPGLVTPV |
| | AQGMGSGAHTADPEKAENVVEPGPPSAKRPKLSSPALSASASDGTDFGSLFDLEH |
| | DLP |
| SPC2: | TCNECKHHVETRWHCTVCEDYDLCITCYNTKNHDHKMEKLGLGLDDESNNQQAAATQ |
| | SPGDSRRLSIQRCIQSLVHACQCRNANCSLPSCQKMKRVVQHTKGCKRKTNGGCPIC |
| | KQLIALCCYHAKHCQENKCPVPFCLNIKQKLRQQQLQHRLQQAQMLRRRMASMQRT |
| | GVVGQQQGLPSPTPATPTTPTGQQPTTPQTPQPTSQPQPTPPNSMPPYLPRTQAAG |
| | PVSQGKAAGQVTPPTPPQTAQPPLPGPPPTAVEMAMQIQRAAETQRQMAHVQIFQRP |
| | IQHQMPP |

Suitable candidate substances also include antibody products (for example, monoclonal and polyclonal antibodies, single chain antibodies, chimeric antibodies and CDR-grated antibodies) which are specific for the first component or the second component, preferably the BOX-I domain of p53 and/or phosphorylated variants thereof. Furthermore, combinatorial libraries, peptide and peptide mimetics, defined chemical entities, oligonucleotides, and natural product libraries may be screened for activity as inhibitors of an interaction between the first component and the second component in assays such as those described below. The candidate substances may be used in an initial screen in batches of, for example 10 substances per reaction, and the substances of those batches which show inhibition tested individually. Candidate substances which show activity in in vitro screens such as those described below can then be tested in in vivo systems, such as mammalian cells which will be exposed to the inhibitor and tested for susceptibility to viral infection or apoptosis as appropriate.

Assays

The assays of the invention may be in vitro assays or in vivo assays, for example using an animal model. One type of in vitro assay for identifying substances which disrupt an interaction between the first component and the second component involves:

contacting a first component, which is immobilised on a solid support, with a non-immobilised second component in the absence of a candidate substance;

contacting the first immobilised component with the non-immobilised second component in the presence of a candidate substance; and

determining if the candidate substance disrupts the interaction between the first component and the second component

Alternatively, the second component may be immobilised and first component non-immobilised.

Binding of the first component to the second component (and vice-versa) may be determined by a variety of methods well-known in the art. For example, the non-immobilised component may be labelled (with for example, a radioactive label, an epitope tag or an enzyme-antibody conjugate). The effect of a candidate substance on an interaction between the two components can be determined by comparing the amount of label bound in the presence of the candidate substance with the amount of label bound in the absence of candidate substance. A lower amount of label bound in the presence of the candidate substance indicates that the candidate substance is an inhibitor of interactions between the first component and the second component.

Alternatively, binding may be determined by immunological detection techniques. For example, the reaction mixture can be Western blotted and the blot probed with an antibody that detects the non-immobilised component. ELISA techniques may also be used.

Candidate substances that are identifiable by the method of the invention as modulating an interaction between a first component and a second component may be tested for their ability to, for example, regulate the cell cycle including apoptosis and growth arrest. Such compounds could be used therapeutically in regulating the cell cycle of a mammalian cell, including preventing cell death in, for example, cell damaged by for example ischemia, or inducing cell death in, for example, neoplastic cells.

Typically, an assay to determine the effect of a candidate substance identifiable by the method of the invention on the regulation of the cell cycle in a mammalian cell comprises:

(a) administering the candidate substance to the cell; and

(b) determining the effect of the candidate substance on the cell cycle, including, for example induction of cell cycle arrest and/or cell death by apoptosis.

Administration of candidate substances to cells may be performed by for example adding directly to the cell culture medium or injection into the cell. The assay is typically carried out in vitro. The candidate substance is contacted with the cells, typically cells in culture. The cells may be cells of a mammalian cell line.

The ability of a candidate substance to induce apoptosis can be determined by administering a candidate compound to cells and determining if apoptosis is induced in said cells. The induction of apoptosis can be determined by various means. There are several techniques known to a skilled person for determining if cell death is due to apoptosis. Apoptotic cell death is characterised by morphological changes which can be observed by microscopy, for example cytoplasmic blebbing, cell shrinkage, intermucleosomal fragmentation and chromatin condensation. DNA cleavage typical of the apoptotic process can be demonstrated using TUNEL and DNA ladder assays.

Alternatively, it may be desired to prevent apoptotic cell death by administering a substance identifiable by the method of the invention. Several techniques known in the art for inducing apoptosis in cells may be used. For example, apoptosis may be induced by stress including UV exposure, growth factor deprivation and heat shock. The ability of the candidate substance to inhibit such apoptosis may be determined by comparing cells exposed to stress in the presence of the candidate substance with those exposed to stress in the absence of the candidate substance.

Therapeutic Uses

The present invention provides a substance capable of modulating an interaction between (i) a p53 polypeptide or a homologue thereof, or a derivative thereof, and (ii) p300 or homologies thereof, or derivatives thereof, for use in a method of regulating the mammalian cell cycle. Typically, said substance may be used to induce cell death, for example in a tumour cell, or to prevent cell death, in for example a cell subject to ischemic damage.

The formulation of a substance according to the invention will depend upon the nature of the substance identified but typically a substance may be formulated for clinical use with a pharmaceutically acceptable carrier or diluent. For example it may be formulated for topical, parenteral, intravenous, intramuscular, subcutaneous, intraocular or transdermal administration. A physician will be able to determine the required route of administration for any particular patient and condition.

Preferably, the substance is used in an injectable form. It may therefore be mixed with any vehicle which is pharmaceutically acceptable for an injectable formulation, preferably for a direct injection at the site to be treated. The pharmaceutically carrier or diluent may be, for example, sterile or isotonic solutions. It is also preferred to formulate that substance in an orally active form.

The dose of substance used may be adjusted according to various parameters, especially according to the substance used, the age, weight and condition of the patient to be treated, the mode of administration used and the required clinical regimen. A physician will be able to determine the required route of administration and dosage for any particular patient and condition.

The present invention will now be further described by way of example and with reference to the Figures which show:

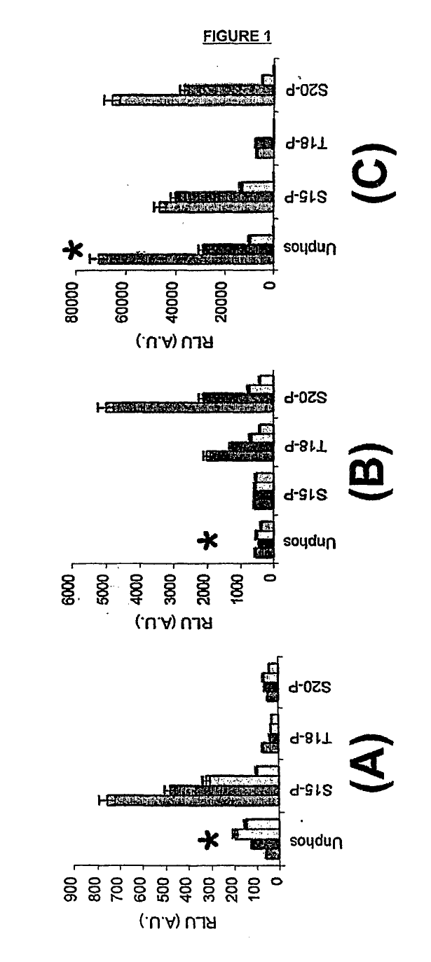

FIG. 1: Phosphorylation stabilises p300-p53 BOX-I peptide complexes. The binding of (A) full-length p300 protein, (B) truncated p300(1135-2414), and (C) full-length Mdm2 protein, to the indicated biotinylated-peptides was analyzed by ELISA as described in the Materials and Methods. The amount of p300 or MDM2 protein bound are represented as RLU=Relative Light Units. The amount of biotinylated peptide titrated onto streptavidin-coated ELISA surfaces is indicated in this Figure, and the other Figures where ELISA is used (* 1 ng,. 0.1 ng, 0.01 ng, 0 ng, respectively).

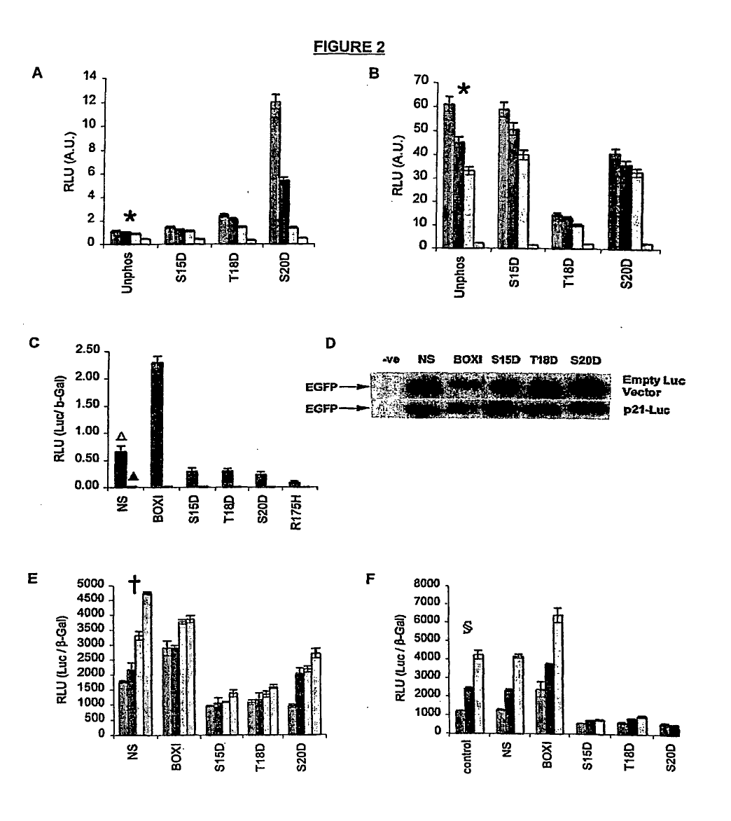

FIG. 2: In vivo inhibition of endogenous p53-dependent transcription by phosphopeptide mimetics. (A) p300 and (B) MDM2 binding in vitro to aspartate-substituted BOX-I domain peptides. The binding of p300 protein and MDM2 protein, to biotinylated-peptides substituted with aspartate at the indicated positions was as described in FIG. 1 (* 1 ng, 0.1 ng, 0.01 ng, 0 ng, respectively). (C). EGFP-Asp¹⁸ and Asp²⁰ peptide fusion proteins inhibit p53-dependent transactivation in vivo. EGFP-constructs or the mutant p53^{HIS175} allele (100 ng, as indicated) were transiently transfected with 2 μg of p21-Luc or 2 μg of control-Luc and 1 μg of control-β-Gal-reporter into cycling A375 cells and the cells harvested 24 hours post-transfection. P53-dependent activity (RLU) is expressed as a ratio of p21-luciferase activity or control-luciferase activity to the internal transfection control [β-Gal] (Δp21-luciferase activity, ▴ control-luciferase). (D). Expression levels of EGFP-peptide fusion proteins in A375 cells. Lysates from cells transfected with the indicated EGFP-peptide fusion constructs, as described in FIG. 2C, were immunoblotted with antibodies to GFP to determine the relative level of each fusion protein expressed in cells transfected with the p21-Luc or 2 μg of control-Luc vectors. (E) p300 can recover p53 activity in cells cotransfected with the inhibitory EGFP-Asp¹⁸ and Asp²⁰ peptide fusion proteins. A375 cells were co-transfected with increasing amounts of the p300 gene and fixed levels of p21-Luc (2 μg), p-Gal-reporter (1 μg), and the EGFP-peptide fusion vectors (100 ng), and the cells were processed for analyzing p53 activity as described in the legend for FIG. 2C († 0 μg, 1 μg, 2 μg and 5 μg, respectively). (F) EGFP-Asp¹⁸ and Asp²⁰ peptide fusion proteins inhibit p53-dependent transactivation in irradiated cells. EGFP-constructs (100 ng, 500 ng, or 1 μg, as indicated) were transiently transfected with 2 μg of p21-Luc or 2 μg of control-Luc and 1 μg of control-β-Gal-reporter into A375 cells that were either cycling, damaged with UV-C, or with ionizing radiation. The cells were processed for analyzing p53 activity as described in the legend for FIG. 2C (§ untreated, UV-C damaged or ionizing radiation).

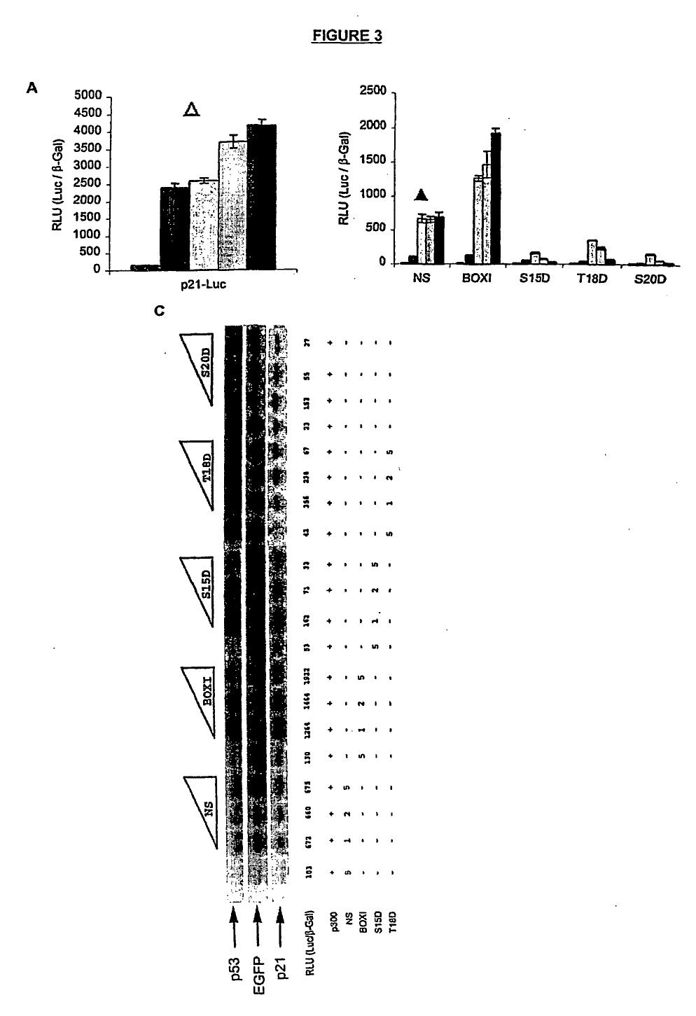

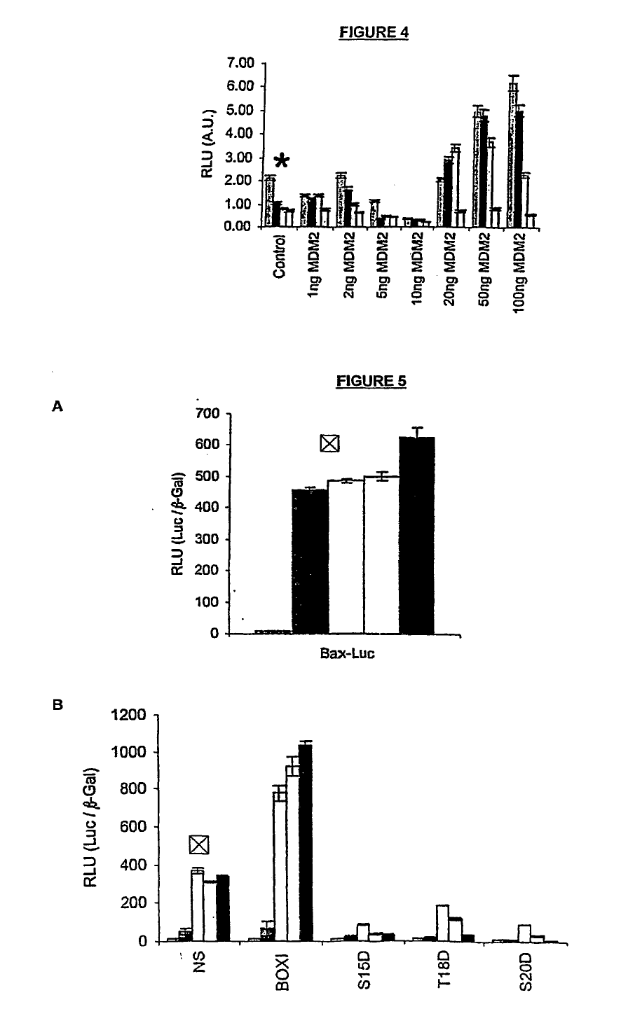

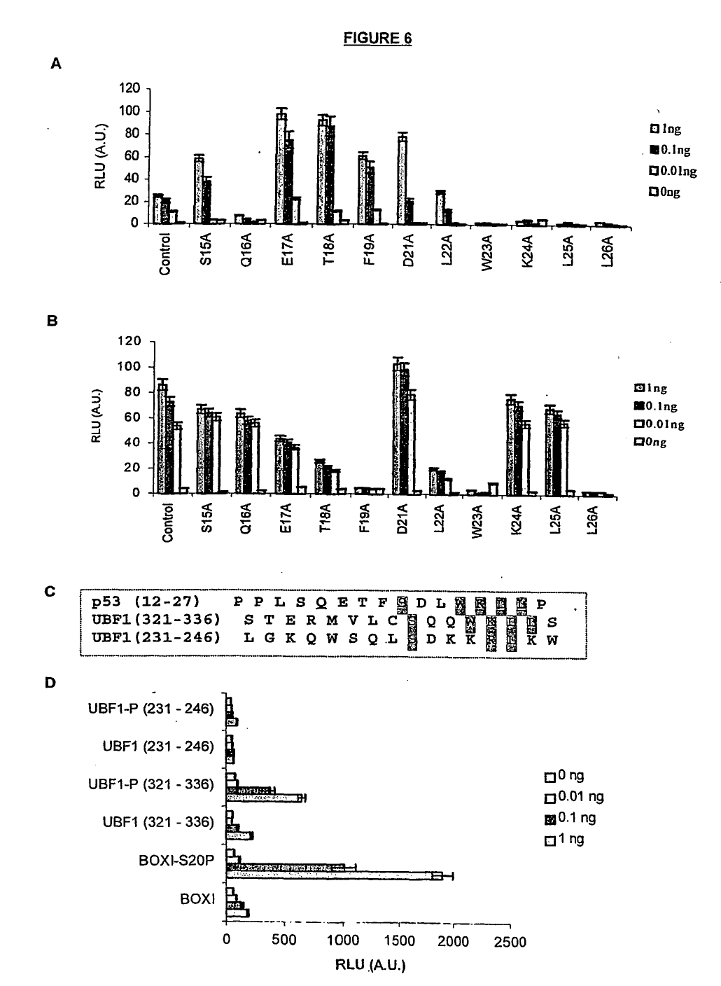

FIG. 3: In vivo inhibition of ectopically expressed p53 from the p21 promoter by phospho-peptide mimetic fusion proteins. (A) Stimulation of p53 activity by cotransfection with p300. Saos-2 cells were transiently co-transfected with 1 μg of pCMV-p53, 2 μg p21-Luc, 1 μg pCMVβ-Gal and increasing amounts of pCMV-βp300. The cells were harvested 30 hours post-transfection and the relative activity is expressed as a ratio of luciferase activity to β-Gal activity (Δ negative control, 0 ng, 1 μg, 2 μg and 5 μg). (B) EGFP-S20D peptide (EPPLSQETFDDLWKL LPENN) inhibits p300 induction of p53-dependent gene expression. Saos-2 cells were transiently co-transfected with 1 μg pCMV-p53, 2 μg p21-Luc, 5 μg pCMVβp300, 1 μg of pCMVβ-Gal and increasing amounts of EGFP-constructs as indicated. The cells were processed as described in the legend of FIG. 3A (▴ control, control, 1 μg, 2 μg and 5 μg). (C) Immunoblots of p53 and EGFP-fusion proteins in transfected Saos-2 cells. Lysates from transfected Saos-2 cells [as described in FIG. 3B] were normalized for protein content by Bradford and loading for immunoblots was confirmed by Red Ponceau staining. The constructs transfected are highlighted by the legend above the Figure (increasing amounts of EGFP fused to NS, BOX-I, S15D, T18D, and S20D BOX-I domain peptides) and are described below the Figure as a “+”. The levels of p53, EGFP, and p21 proteins are in the top, middle, or bottom panel, respectively. p53-dependent activity (in RLU's) from FIG. 3B is listed underneath the immunoblots for direct comparison of p53 activity to p53 protein and EGFP protein levels.

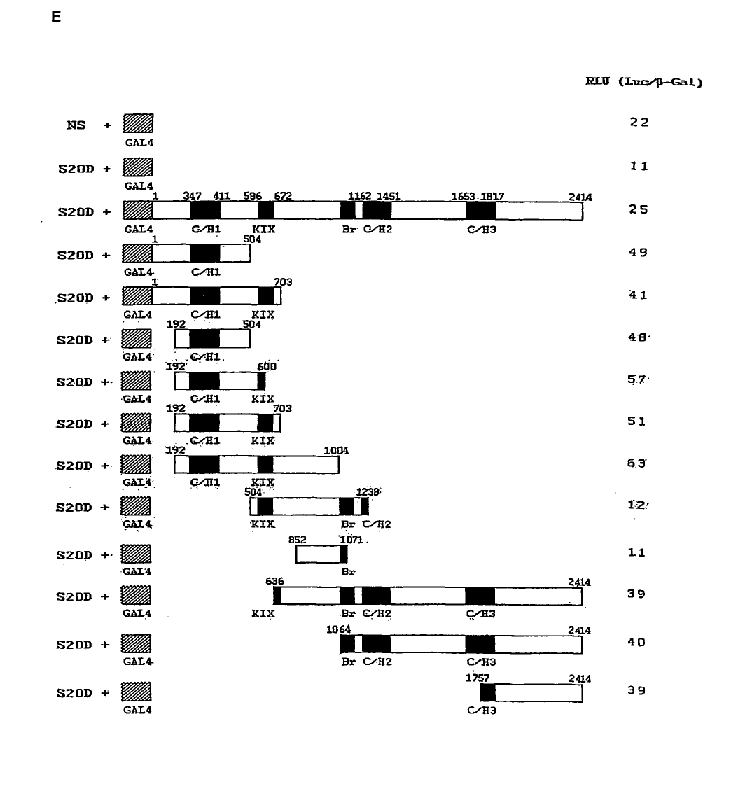

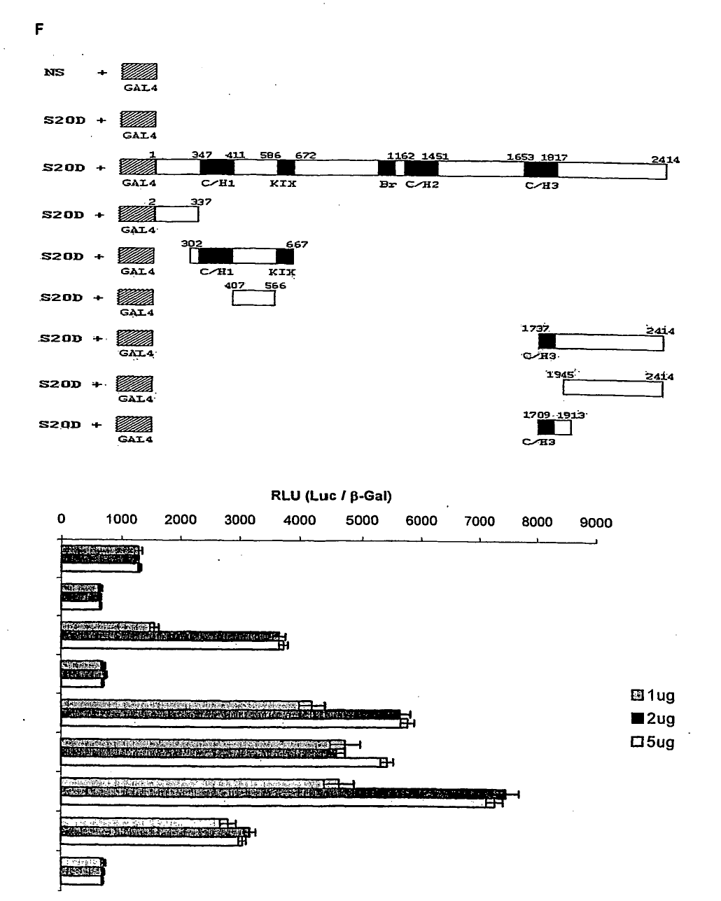

FIG. 4: MDM2 protein can compete with p300 binding to the Ser²⁰ phospho-peptide ligand. The ability of MDM2 to compete for p300 binding to the Ser²⁰ phospho-peptide ligand was examined in order to determine the relative affinities of each protein for the peptide ligand. The binding of full-length p300 protein (1 ng; control panel) to increasing amounts of biotinylated peptide titrated onto streptavidin-coated surfaces was incubated alone or with increasing amounts of MDM2 protein from 1 ng to 100 ng, as indicated. At 5 to 10 fold excess levels of MDM2 protein over p300 protein (from 5 to 10 ng), a 50% to 75% inhibition of p300 binding to its ligand can be achieved. However, at higher concentrations of MDM2 protein (from 50 to 100 ng), a very strong binding signal can be achieved with p300, presumably due to the ability of p300 to bind to the MDM2 protein-Ser²⁰ phospho-peptide complex in the ELISA well. The MDM2-p300 complex has been reported previously (Grossman et al., 1998). Although these data indicate that MDM2 and p300 protein binding affinities to the Ser²⁰ phospho-peptide is within an order of magnitude, the competing p300-MDM2 protein interaction complicates the precise quantitation of the relative affinities of each protein for this peptide ligand (* 1 ng, 0.1 ng, 0.01 ng and 0 ng).

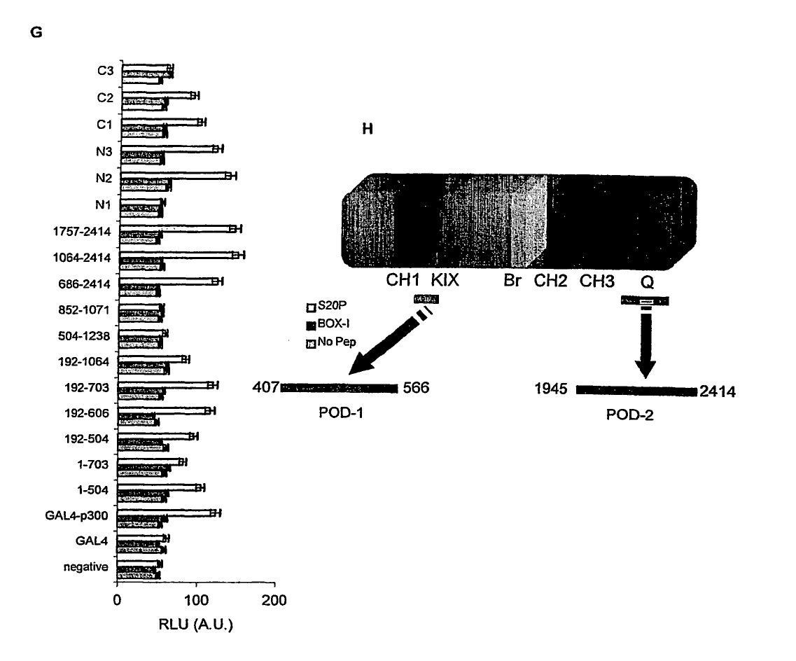

FIG. 5: In vivo inhibition of ectopically expressed p53 activity from the bax promoter by phospho-peptide mimetic fusion proteins. (A) Stimulation of p53 activity by cotransfection with p300. Saos-2 cells were transiently co-transfected with 1 μg of pCMV-p53, 2 μg bax-Luc, 1 μg pCMVβ-Gal and increasing amounts of pCMV-βp300. The cells were harvested 30 hours post-transfection and the relative activity is expressed as a ratio of luciferase activity to β-Gal activity. (B) EGFP-S20D peptide fusion protein inhibits p300 induction of p53-dependent gene expression. Saos-2 cells were transiently co-transfected with 1 μg pCMV-p53, 2 μg bax-Luc, 5 μg pCMVβp300, 1 μg of pCMVβ-Gal and increasing amounts of EGFP-S20D or EGFP-control. Cells were harvested 30 hours post-transfection and relative activity is expressed as a ratio of luciferase activity to β-Gal activity ( [symbol] control, 0 μg, 1 μg, 2 μg and 5 μg).

FIG. 6: Definition of the p300 Binding Specificity within the BOX-I domain of p53 and identification of POD-1/2. The binding of: (A) Full-length p300 protein and (B) full-length MDM2 protein, to biotinylated-Ser²⁰ phospho-peptides substituted with alanine at the indicated positions was analysed by ELISA. The amount of biotinylated peptide-titrated onto streptavidin-coated surfaces is as indicated. The amount of p300 or MDM2 protein bound is quantitated as luciferase activity (RLU) using a peroxidase-linked secondary-antibody coupled to anti-p300 or anti-MDM2 antibodies. (C) The BOX-I domain from amino acids 12-27 highlight the amino acids required for p300 binding (underlined). The alignment with UBF1 forming a consensus p300-binding site is also illustrated with consensus residues marked in grey. (D) The binding of p300 to UBF peptides or the p53-BOX-1 peptide with or without a phosphate substitution at the highlighted serine residue analysed by ELISA. The amount of biotinylated peptide titrated onto streptavidin-coated surfaces is as indicated. The amount of p300 protein bound is quantitated as luciferase activity (RLU) using a peroxidase-linked secondary-antibody coupled to anti-p300 or anti-MDM2 antibodies. (E) and (F) Identification of EGFP-S20D binding domains (POD-1/2) using an in vivo peptide-binding assay. 5 μg EGFP-S20D was co-transfected with 5 μg of the indicated GAL4-p300 constructs into cycling A375 cells with 1 μg p21-Luc and pCMVβ-Gal. To function as a background control, 5 μg EGFP-NS (ELKLRILQSTVPRARDPPL) was co-transfected with 5 μg GAL4. The data are represented as reporter Luciferase activity (RLU) normalized to β-gal activity. (G) Identification of EGFP-S20D binding domains (POD-1/2) using an in vitro p300-peptide-binding assay. HCT116 p53−/− cells were transfected with 5 μg of GAL4-p300 and lysates captured with an anti-GAL4 antibody. The indicated biotinylated peptide (BOX-1, Ser²⁰-phospho-BOX-I, or no peptide) was added and the amount of p300 protein bound is quantitated as luciferase activity (RLU) using a peroxidase-linked secondary-antibody coupled to anti-p300 or anti-MDM2 antibodies. (H) Schematic diagram illustrating the Ser²⁰-phosphate binding regions of p300; POD-1 (Phospho-Serine 20 Interacting Domain 1) and POD-2 ((Phospho-Serine 20 Interacting Domain 2); relative to the other well-characterized domains including CH1/CH3, KIX, Bromo, CH2, and Q.

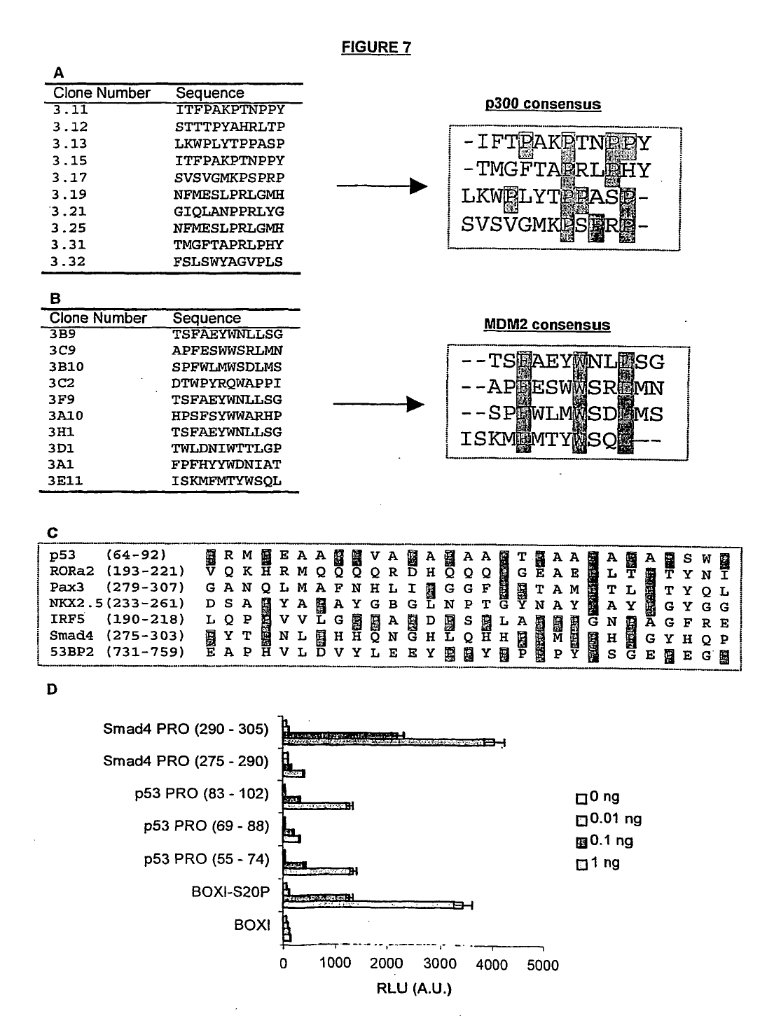

FIG. 7: Identification of a novel p300-binding motif by phage-peptide display. (A) Identification of p300-binding motifs by phage-peptide display. The isolated clones from full-length recombinant p300 as a target protein for phage-peptide display are highlighted with the consensus motifs in grey. (B) As a control to define library integrity, we also identified canonical MDM2-binding motifs by phage-peptide display. The isolated clones from MDM2 as a target protein for phage-peptide display are highlighted with the consensus motifs in grey. (C) Sequence alignments of proline-rich regions from the peptides for potential p300 docking sites in open reading frames derived from transcription factors from the database. Highlighted residues (in grey) are within a PXP, PXXP or PXXXP motif. (D) p300 binds to a subset of polyproline peptides from p53 and SMAD4. Biotinylated peptides were used as a target for p300 in an ELISA containing peptide sequences from the aligned Smad4 and p53 proline rich regions. Amount of peptide incubated is as indicated. The amount of p300 protein bound is quantitated as luciferase activity (RLU) using a peroxidase-linked secondary-antibody coupled to anti-p300 or anti-MDM2 antibodies.

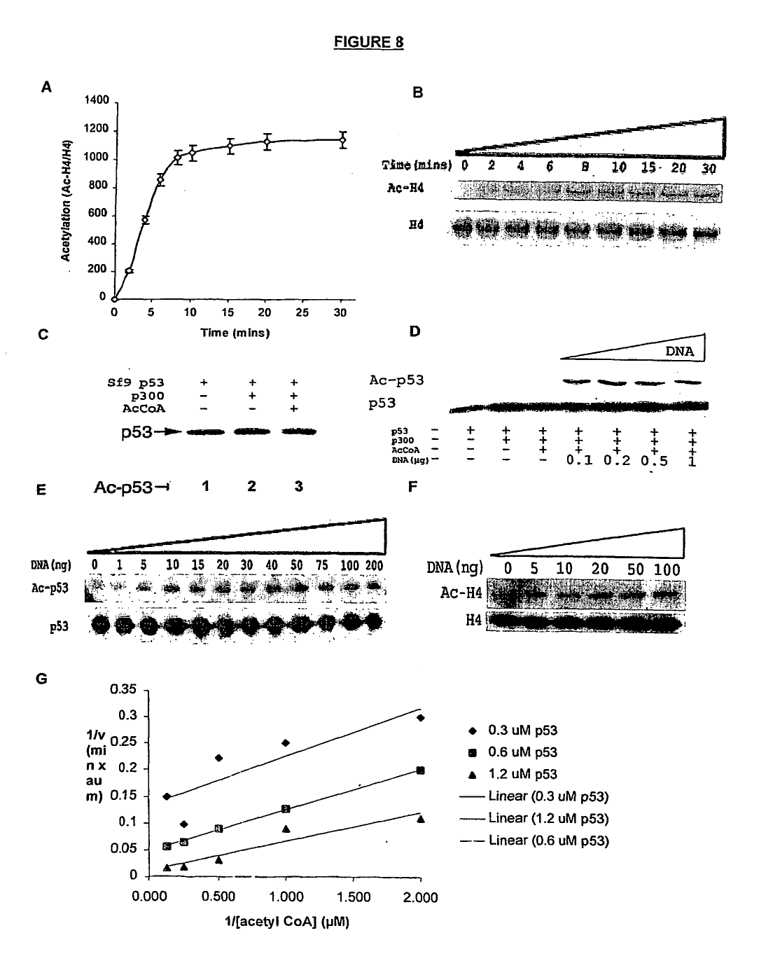

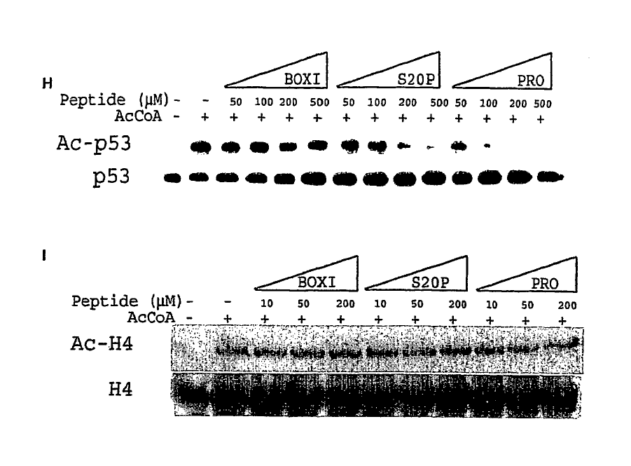

FIG. 8. p300-mediated p53 acetylation is stimulated by DNA and is inhibited by either polyproline and Ser²⁰-phospho BOX-I peptides derived from p53. (A) Monophasic kinetics of acetylation using purified p300 and the histone H4 substrate. 100 ng of purified p300 was incubated over the indicated times (0 minutes to 30 minutes) with 1 μg of histone H4 and acetyl-COA and relative acetylation over time determined by bioluminescence or (B) western blot. (C) Acetylation of p53 by p300. 100 ng of p300 and 400 ng of p53 was incubated for 10 minutes and relative acetylation determined by western blot using an anti-p53-acetylation antibody: p53 alone (lane 1), p53+p300 (lane 2), and p53+p300+acetyl-CoA (lane 3). (D) and (E) p53 acetylation by p300 is stimulated by the addition of p53 consensus site DNA. Acetylation reactions were carried out exactly as described in (C) with the addition of varying amounts of p53 consenus site DNA (as indicated). (F) DNA does not stimulate histone acetylation by p300. Acetylation reactions were carried out as described in (A) but with the addition of p53 consensus site DNA and for a reaction time of 6 minutes. (G) p300 acetylation of p53 follows a ping-pong mechanism. Acetylation reactions were carried out as before but with varying concentrations of acetyl-CoA at different concentrations of p53 as indicated and a resultant double-reciprocal plot was drawn. (H) p300-dependent acetylation of p53 is inhibited by the p300 polyproline-binding peptide derived from p53 (55-74). Acetylation reactions were carried out as before except with the addition of varying concentrations of peptides: BOX-I; Ser²⁰Phospho-BOX-I or polyproline (55-74). The top panel measures acetylated p53 and the bottom panel measures total p53 protein levels. (I) Histone acetylation is not inhibited by the p300 polyproline-binding peptide derived from p53. Acetylation reactions were carried out as in (H) but with 1 μg of histone H4 as the substrate for p300. The insensitivity of histone acetylation to the p300 polyproline-binding peptide derived from p53 indicates that p53 acetylation inhibition by this peptide is not allosteric.

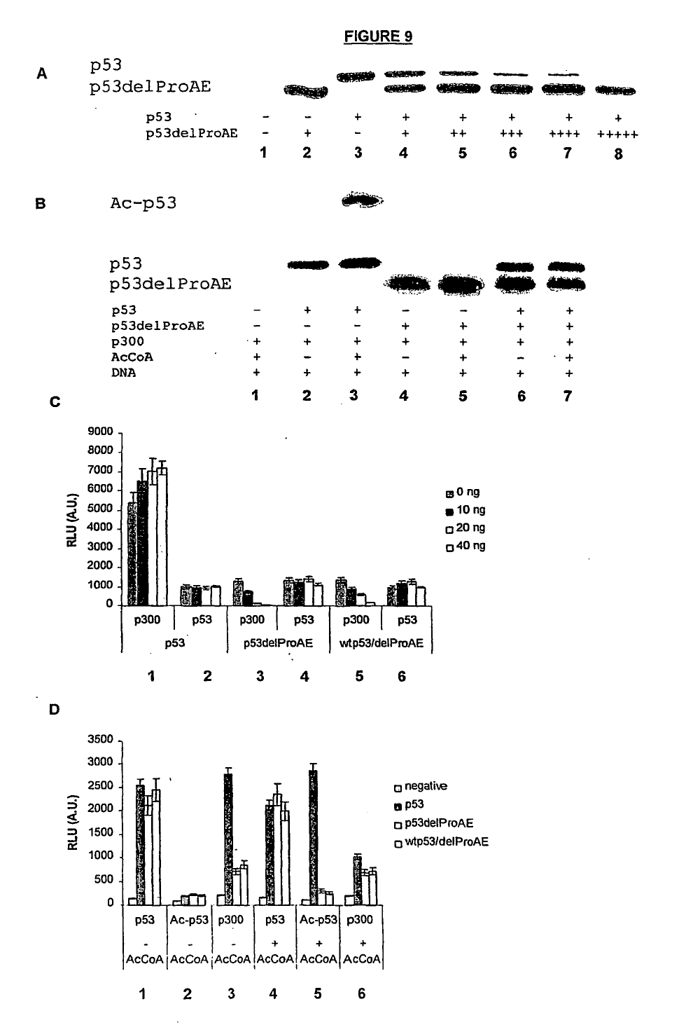

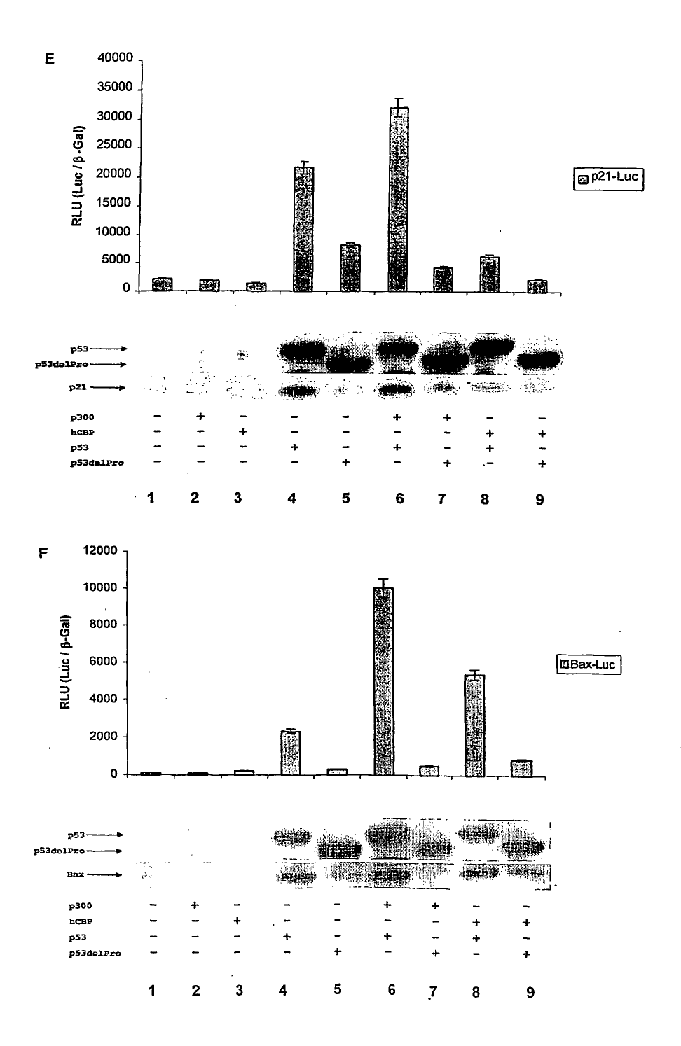

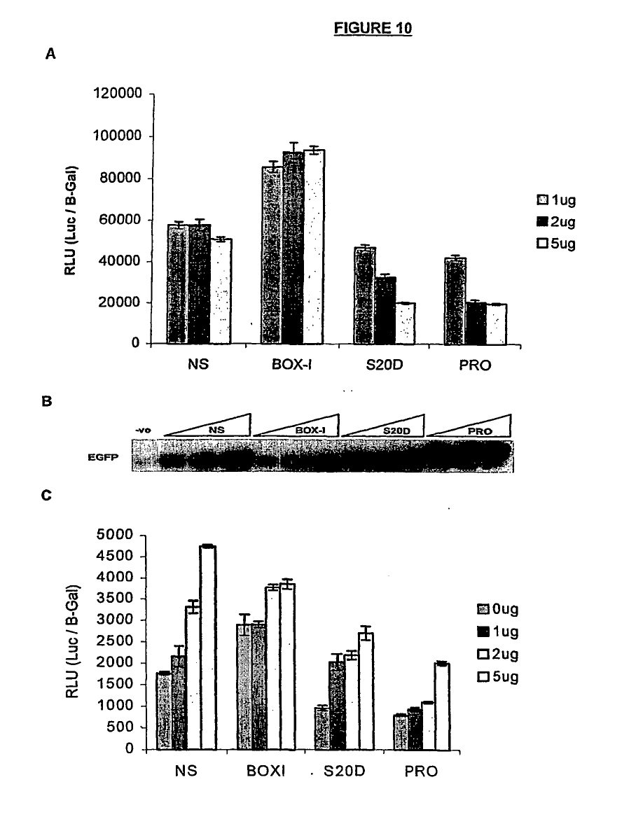

FIG. 9: The polyproline domain of p53 is critical for p300 binding and acetylation. (A) Expression of recombinant human p53, p53ΔProAE and p53/p53ΔProAE mixed tetramers in Sf9 cells. Baculovirus harbouring p53ΔProAE was infected at an increasing titre whilst keeping wild-type p53 constant. The amount of virus where equal levels of mixed tetramer were produced determined by western blot with anti-p53 were then scaled up for purification of the mixed tetramer. (B) Strict requirement for the polyproline domain of p53 for DNA-dependent acetylation by p300. Acetylation reactions were carried out as before (FIG. 8E) but with the addition of wild-type p53, p53ΔProAE or the p53/p53ΔProAE mixed tetramer. Resultant acetylation was measured using anti-acetylation-p53 antibodies (top panel) and normalised to total p53 protein (bottom panel). (C) p300 binding to p53 is compromised by the deletion of the polyproline domain. P53, p53ΔProAE or p53/p53ΔProAE were captured on the solid-phase with ICA-9 anti-p53 monoclonal antibody including a titration of p53 consensus site DNA as indicated in the panel (0, 10, 20, or 40 ng). After this, the captured p53 isoforms were incubated with buffer lacking p300 (panels 2, 4, and 6) or with p300 (panels 1, 3, and 5). The relative binding of p300 to the p53 isoforms (panels 1, 3, and 5) was determined using anti-p300 antibodies and quantitated as RLU. The relative levels of the p53 isoforms (panels 2, 4, and 6) were determined using anti-p53 antibodies (CM-1) and quantitated as RLU. A dose-dependent decrease in p300-binding with increasing DNA was observed using the p53ΔProAE or p53/p53ΔProAE isoforms (panels 3, and 5), while wild-type p53-p300 was stabilized slightly by increasing DNA titrations (panel 1). (D) Acetylation of p53 by p300 reduces the affinity of p53 for p300 in a polyproline-dependent manner. Complete acetylation reactions were assembled without acetyl-CoA (panels 1-3) or with Acetyl-CoA (panels 4-6) and carried out as before using the different p53 isoforms (as indicated in the Figure) which were captured by the anti-p53 antibody ICA-9. Each reaction was probed for: p53 protein levels using CMI (panels 1 and 4), p300 protein levels using anti-p300 IgG (panels 3 and 6), and for acetylation of p53 using anti-Ac-p53 IgG (Panels 2 and 5). The binding or acetylation data are quantitated as RLU's. (E) and (F) The polyproline domain of p53 is required for efficient transactivation of the p21 and bax promoter. The transactivation activity of p53 and p53ΔProAE on the (E) p21 and (F) bax promoters (RLUs) is expressed as a ratio of p21-Luc or Bax-Luc to the internal transfection control (pCMVβ-Gal). Expression levels of p53 protein, and endogenous p21 protein and Bax protein were quantitated by western blotting. In each transfection, 1 μg of pCMV-p53 or pCMV-p53ΔProAE alone or with 5 μg pCMV-β300 or pCMV-hCBP was added as indicated by − or +.

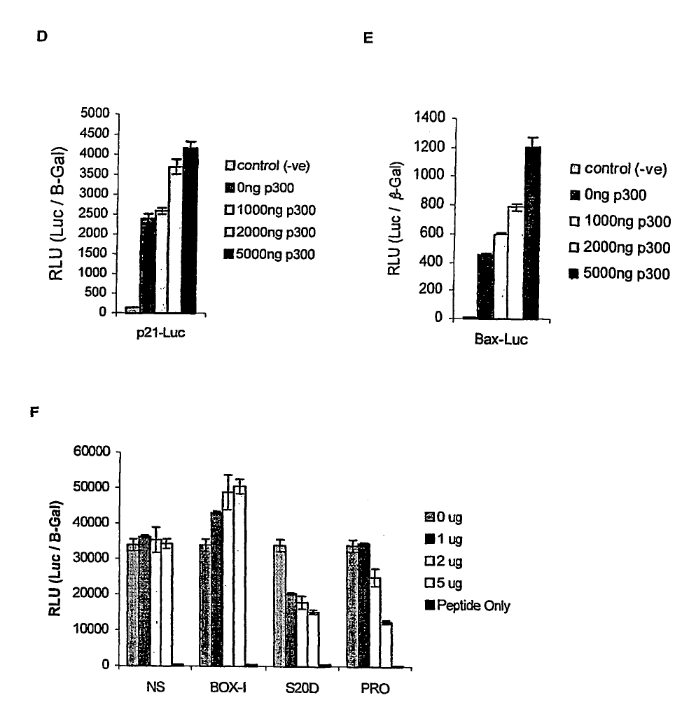

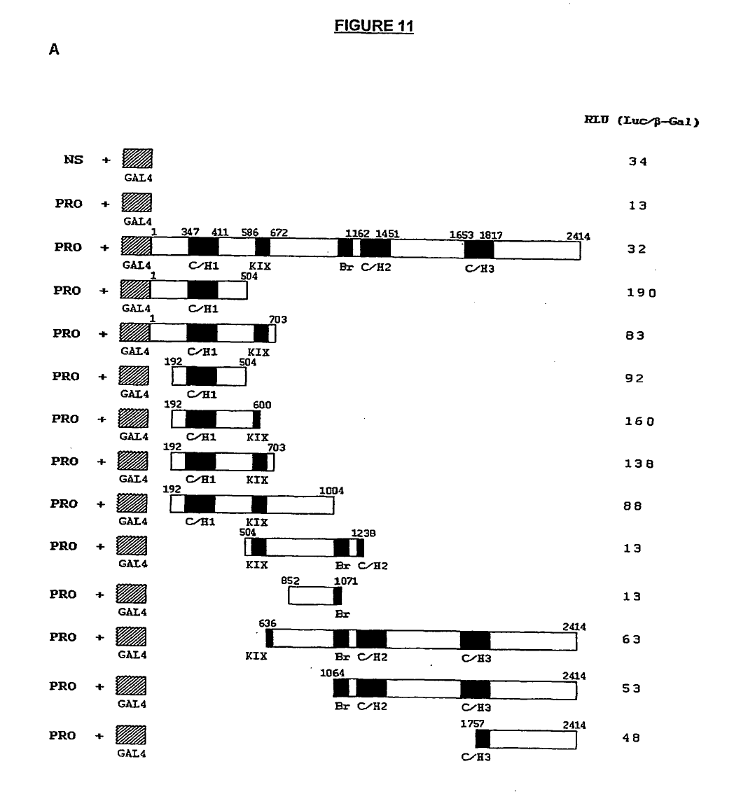

FIG. 10: In vivo inhibition of p53-p300 dependent transcription by a polyproline peptide. (A) EGFP-PRO inhibits p53-dependent transactivation in vivo. EGFP-peptide fusion constructs 1 μg, 2 μg and 5 μg were transfected with 1 μg p21-Luc and 1 μg CMVβ-Gal into cycling A375 cells. RLUs were calculated as described in FIG. 9E. (B) Expression levels of EGFP-peptide fusion proteins. Lysates from transfected cells, from FIG. 10A, were immunoblotted with antibodies to EGFP. (C) p300 can recover transcriptional inhibition from EGFP-PRO. A375 cells were co-transfected with 5 μg of EGFP-peptide constructs and increasing amounts of CMVβ-p300 (0 μg, 1 μg, 2 μg and 5 μg) and p21-Luc. (D) and (E) Stimulation of p53 activity by co-transfection with p300 on (D) p21 and (E) bax promoter. Saos2 cells were co-transfected with 1 μg pCMV-p53, p21-Luc, pCMV-β-Gal and increasing amounts of pCMVβ-p300 (0 μg, 1 μg, 2 μg and 5 μg). As a negative control, 5 μg pCMVp-p300 only was co-transfected with the reporter constructs. (F) and (G) EGP-PRO inhibits p53-p300 co-stimulation on p21 and bax promoters, respectively. Saos2 cells were co-transfected with 1 μg pCMV-p53, 5 μg pCMVβ-p300 and reporter constructs as in FIG. 10D with an increasing amount of EGFP-peptide constructs (0 μg, 1 μg, 2 μg and 5 μg). As a control, 5 μg EGFP construct and 5 μg pCMVβ-p300 with the reporter constructs were co-transfected as indicated.

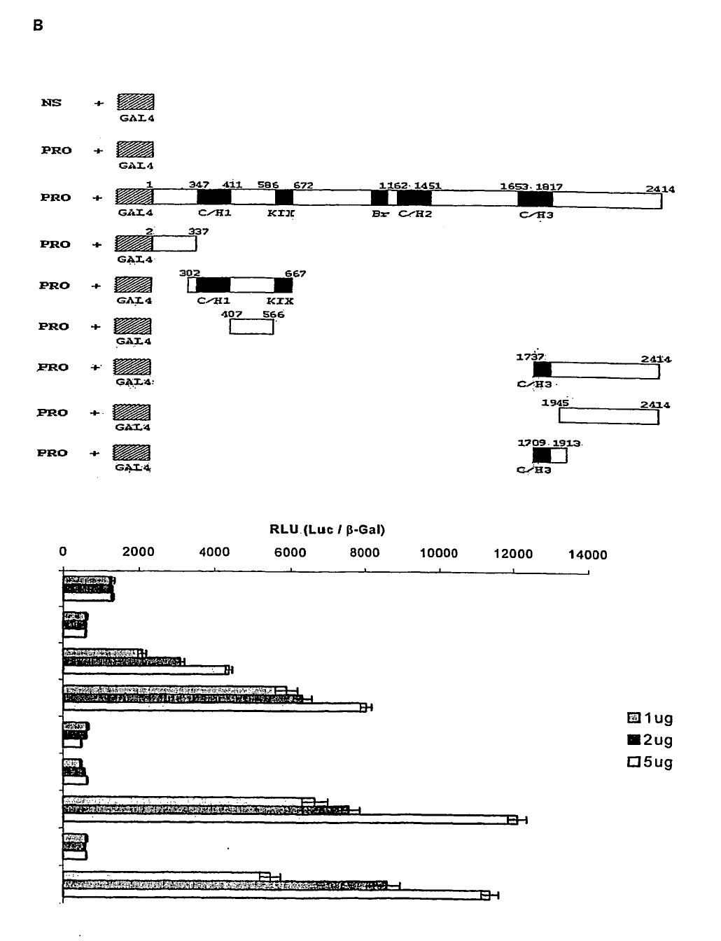

FIG. 11: Identification of p300 site for polyproline contact domains SPC-1/2. (A) and (B) Identification of EGFP-PRO binding domains (SPC-1/2) using an in vivo peptide-binding assay. 5 μg EGFP-PRO was co-transfected with 5 μg of the indicated GAL4-p300 constructs into cycling A375 cells with 1 μg p21-Luc and pCMVβ-Gal. To function as a background control, 5 μg EGFP-NS was co-transfected with 5 μg GAL4. The data are represented as reporter Luciferase activity (RLU) normalized to β-gal activity. (C) Identification of EGFP-PRO binding domains (SPC-1/2) using an in vitro p300-peptide-binding assay. HCT116 p53−/−cells were transfected with 5 μg of GAL4-p300 and lysates captured with an anti-GAL4 antibody. The indicated biotinylated peptide (BOX-I, polyproline-peptide (55-74), or no peptide) was added and the amount of p300 protein bound is quantitated as luciferase activity (RLU) using a peroxidase-linked secondary-antibody coupled to anti-p300 or anti-MDM2 antibodies. (D) Schematic diagram illustrating the Ser²⁰-antibody coupled to anti-p300 or anti-MDM2 antibodies. (D) Schematic diagram illustrating the Ser²⁰-phosphate binding regions of p300; SPC-1 and SPC-2; relative to the other domains including POD-1, POD-2, CH1/CH3, KIX, Bromo, CH2, and Q. EGFP-PRO peptide (DEAPRMPEAAPPVAPAPAAPTPAAPAPAPSWPL) selectively induces a G2/M arrest independent of p53 status. EGFP-S20D and EGFP-PRO selectively induce a G2/M arrest in p53^{+/+} cells. (E) and (G) 5 μg EGFP-NS, BOX-I, S20D or PRO were transfected into HCT116^{(p53+/+)} cells along with 1 μg pCMV-CD20 and transiently transfected population detected by CD20-FITC antibody with the resultant cell cycle profile analysed by a FACScan (Becton Dickinson). (F) and (H) 5 μg EGFP-NS, S20D or PRO were transfected and processed as in (A) but with HCT116 p53−/− cells.

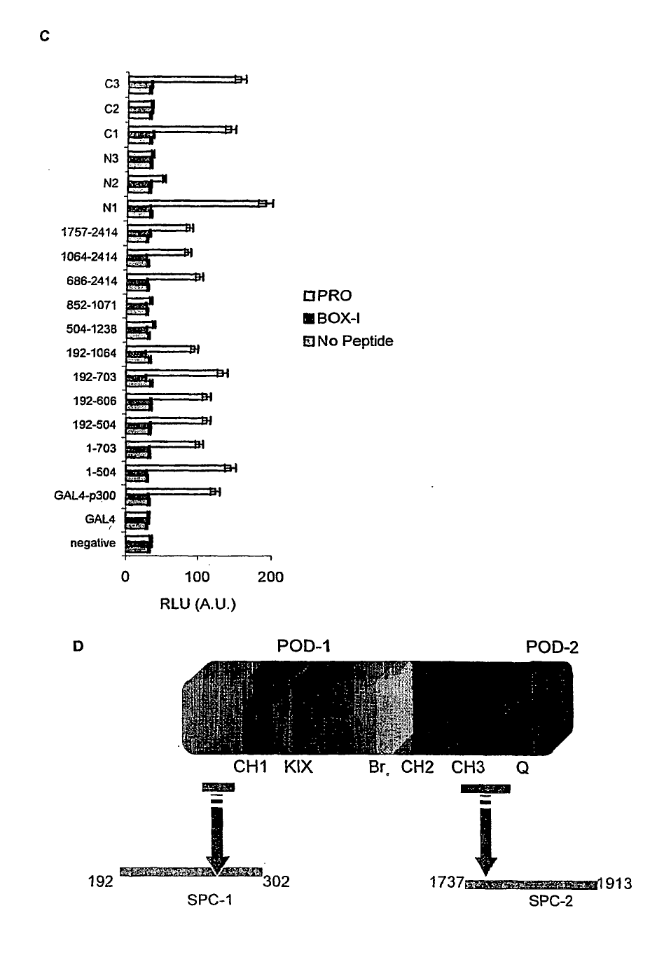

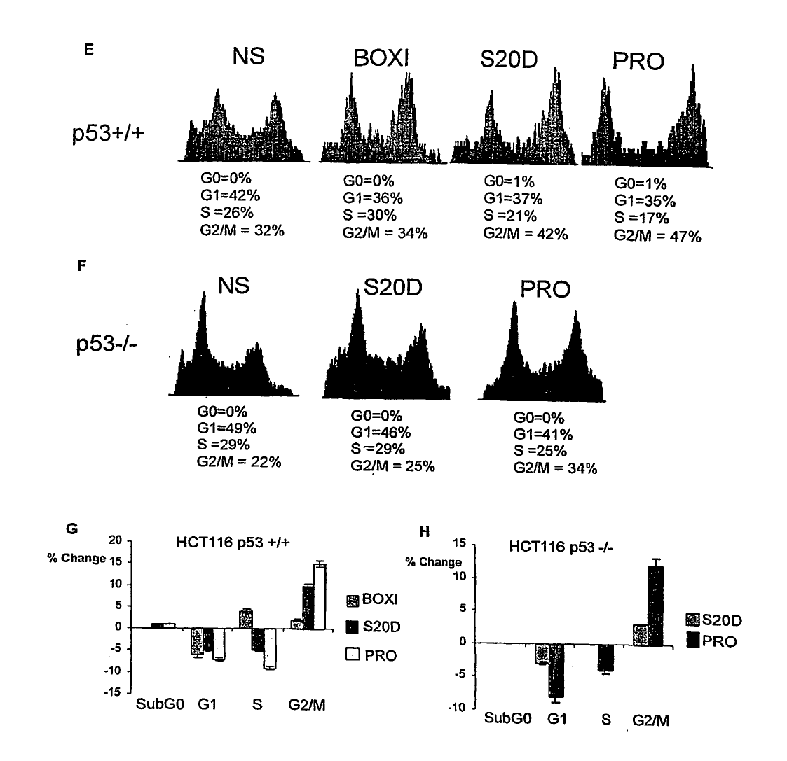

FIG. 12. Stages in the assembly of a p300-p53 oligomeric protein complex. (Stage 1) p300 docks via its SPC-1/2 and POD-1/2 domains onto the BOX-I domain of p53 and the polyproline domain of p53 (FIGS. 6 and 11). p300 is tetravalent with respect to p53 docking and p53 is octavalent with respect to p300 binding, so it is not clear whether this docking involves intra or interdomain interactions. (Stage 2) p300 acetylation is sequential, ordered, and requires first the formation of a high energy acetyl˜p300 complex prior to protein substrate binding (ping-pong mechanism, FIG. 8G and (Thompson, Kurooka et al. 2001)). (Stage 3) The acetylation of p53-DNA complexes by p300 requires the polyproline domain of p53 (FIG. 9C) and the SPC-1/2+POD-1/2 domains of p300 (FIG. 8H). (Stage 4) Dissociation of the p300-p53 complex is acetyl-CoA dependent and polyproline-binding dependent (FIG. 9). This model is consistent with recent data showing that the C-terminal acetylation motif of p53 is a positive regulatory domain and is required for p300-driven p53-dependent transcription in vitro (Espinosa and Emerson 2001) and that acetylation may function a positive signal for some transcription factors to recruit or renew co-activator complexes (Prives and Manley 2001).

### EXAMPLE 1

Results and Discussion

phosphate binding regions of p300; SPC-1 (Site for Polyproline Contact 1) and SPC-2 (Site for Polyproline Contact 2); relative to the other domains including POD-1, POD-2, CH1/CH3, KIX, Bromo, CH2, and Q. EGFP-PRO peptide (DEAPRMPEAAPPVAPAPAAPTPAA PAPAPSWPL) selectively induces a G2/M arrest independent of p53 status. EGFP-S20D and EGFP-PRO selectively induce a G2/M arrest in p53^{+/+} cells. (E) and (G) 5 μg EGFP-NS, BOX-I, S20D or PRO were transfected into HCT116^{(p53+/+)} cells along with 1 μg pCMV-CD20 and transiently transfected population detected by CD20-FITC antibody with the resultant cell cycle profile analysed by a FACScan (Becton Dickinson). (F) and (H) 5 μg EGFP-NS, S20D or PRO were transfected and processed as in (A) but with HCT116 p53−/− cells.

FIG. 12. Stages in the assembly of a p300-p53 oligomeric protein complex. (Stage 1) p300 docks via its SPC-1/2 and POD-1/2 domains onto the BOX-I domain of p53 and the polyproline domain of p53 (FIGS. 6 and 11). p300 is tetravalent with respect to p53 docking and p53 is octavalent with respect to p300 binding, so it is not clear whether this docking involves intra or interdomain interactions. (Stage 2) p300 acetylation is sequential, ordered, and requires first the formation of a high energy acetyl-p300 complex prior to protein substrate binding (ping-pong mechanism, FIG. 8G and (Thompson, Kurooka et al. 2001)). (Stage 3) The acetylation of p53-DNA complexes by p300 requires the polyproline domain of p53 (FIG. 9C) and the SPC-1/2+ POD-1/2 domains of p300 (FIG. 8H). (Stage 4) Dissociation of the p300-p53 complex is acetyl-CoA dependent and polyproline-binding dependent (FIG. 9). (Stage 5) The p53 and p300 are recyled and p53 binds the target promoter once more. This model is consistent with recent data showing that the C-terminal acetylation motif of p53 is a positive regulatory domain and is required for p300-driven p53-dependent transcription in vitro (Espinosa and Emerson 2001) and that acetylation may function a positive signal for some transcription factors to recruit or renew co-activator complexes (Prives and Manley 2001).

### EXAMPLE 1

Results and Discussion

Full-length p300 binds preferentially to small phospho-peptides derived from the BOX-I domain of p53.

The BOX-I domain is subject to two key protein-protein interactions with cellular components: MDM2 and p300. There are unresolved data demonstrating that either Thr¹⁸ and/or Ser²⁰ phosphorylation events have no affect on p53 activity (Ashcroft et al., 1999) or that Ser²⁰ phosphorylation is required for p53 activity (Unger et al., 1999). The present inventors therefore wished to determine amongst other things if covalent modification at the phospho-acceptor sites (Thr¹⁸ or Ser²⁰) had the most striking affect on MDM2 binding to p53 and/or p300 binding to p53. Full-length MDM2 and p300 proteins were produced and used in an ELISA based peptide binding assay for quantitating the specific activity of MDM2 and p300 proteins. A titration of the peptide phosphorylated at Ser²⁰ did not inhibit p300 interaction with the BOX-I domain, but rather promoted a striking stabilization of the p300-peptide complex, which is in contrast with the inability of p300 protein to bind at all to the unphosphorylated BOX-I domain peptides (FIG. 1A). The Thr¹⁸ phospho-peptide similarly displayed a significant binding affinity for p300 protein, but not with the same potential observed with the Ser²⁰ phospho-peptide (FIG. 1A).

Surprisingly, full-length p300 protein exhibited a relatively low affinity for the Ser¹⁵ phospho-peptide to full-length p300 protein (FIG. 1A) despite previous observations demonstrating that phosphorylation of p53 by DNA-PK increased the acetylation by CBP in vitro (Lambert et al., 1998). These differences may simply reflect that K_{d} alterations of full-length p300 towards small phospho-peptides and full-length phosphorylated p53 protein, as at higher concentrations of full-length p300 protein in the ELISA, Ser¹⁵ phospho-peptide binding was observed (data not shown). Additionally, an N-terminal deletion of p300 protein to produce the variant p300(1135-2414) stabilized the binding of the protein to the Ser¹⁵ phospho-peptides (FIG. 1B). More strikingly, the deleted variant of p300 protein abrogates its ability to bind to the Ser²⁰ and Thr¹⁸ phospho-peptides (FIG. 1B), suggesting that the N-terminal domain of p300 contains a regulatory motif that is essential for stabilizing the binding of p300 to the Ser²⁰ and Thr¹⁸ phosphorylated BOX-I region. Under conditions where neither full-length p300 or p300 (1135-2414) harboured the ability to bind the unphosphorylated BOX-I domain peptide, full-length MDM2 protein bound with highest affinity to the unphosphorylated BOX-I domain peptide (FIG. 1C). As reported previously (Craig et al., 1999b; Sakaguchi et al., 2000), the most potent inhibitor of MDM2 binding was the Thr¹⁸ phospho-substitution, while the Ser¹⁵ or Ser²⁰ phospho-substituted peptides displayed the most ineffective inhibition of MDM2 protein binding (FIG. 1C). MDM2 and p300 proteins display similar affinities for the Ser²⁰ phospho-substituted peptide in a direct peptide-competition binding assay when equivalent amounts of protein are titrated (FIG. 4), suggesting that phosphorylation may serve as a regulatory switch for discrimination between p300 and MDM2 binding. Together, these data highlight the requirement for the BOX-I domain to be phosphorylated at Ser²⁰ or Thr¹⁸ in order for p300 protein in its full-length state to acquire its highest specific activity.

BOX-I Domain Phospho-Mimetic Peptides Selectively Inhibit p53-Dependent Transcription In Vivo.

The biochemical data now suggest that the primary role of phosphorylation of p53 at Ser²⁰ is to stabilize the p300-p53 complex (FIG. 1), rather than the direct inhibition of MDM2 protein binding, based on the diametrically opposed specificity of the BOX-I domain of p53 towards MDM2 and the Ser²⁰-phosphorylated BOX-I domain towards p300. As site directed mutagenesis of p53 has often failed to support a clear role for Ser²⁰, Thr¹⁸, or even Ser¹⁵ phosphorylation in promoting p53 activity (Ashcroft et al., 1999), we took an alternative approach to determine whether this cluster of phosphorylation events affects p300 or MDM2 protein function in vivo by developing small phospho-peptide BOX-I mimetics for intracellular expression. In order to produce a phospho-peptide mimetic for in vivo use, phosphorylation was mimicked by the use of aspartate-substituted peptides at the Ser¹⁵, Thr¹⁸ or Ser²⁰ residues. P300 binding to the BOX-I domain was stabilized by the Asp²⁰ or Asp¹⁸ substitutions (FIG. 2A), while MDM2 protein was most inhibited by the Asp¹⁸ substitution mutant (FIG. 2B). The specific activity of full-length p300 protein (in RLU's) in binding to phospho-peptides or aspartate-substituted peptides can be compared directly (FIG. 1A vs FIG. 2A) and it is clear that the aspartate-substitution does not fully compensate for the phosphate moiety. Nevertheless, these data indicate that a single point mutation in the BOX-I motif can convert the domain into a p300-binding ligand, highlighting the suitability of the use of aspartate mutants in vivo as relatively effective phosphate mimics.

A series of phospho-peptide mimetics were then constructed by fusion with EGFP for intracellular synthesis to determine whether the peptides can affect transcription from a p53-dependent reporter gene. EGFP-BOX-I domain phospho-peptide mimetics (EPPLSQETFSDLWKLLPENN) were produced by incorporating a gene encoding the amino acids 11-30 of human p53, incorporating the BOX-I domain, with either no modifications (EGFP-BOX-1) or an aspartate substitution at Ser¹⁵ (EGFP-S¹⁵D), Thr¹⁸ (EGFP-T18D) or Ser²⁰ (EGFP-S20D) fused to the C-terminus of EGFP-NS. Proliferating A375 cells were transiently co-transfected with EGFP-NS, EGFP-BOX-I, EGFP-S15D, EGFP-T18D or EGFP-S20D and control or p21-Luciferase reporter constructs. Changes in the basal p53-dependent transcription activity was quantitated 24 hours post-transfection. All three of the EGFP-aspartate-substituted fusion proteins inhibited basal p53-dependent transactivation of the p21 promoter relative to controls, with the EGFP-S20D inhibiting p53 activity to the highest degree (FIG. 2C). EGFP-S15D and EGFP-T18D fusion proteins both reduced significantly the basal p53-dependent transactivation of the p21 promoter (FIG. 2C). As a control for comparison, the dominant negative mutant p53 encoded by the HIS175 allele gave rise to the highest level of inhibition of p53-dependent activity (FIG. 2C). The EGFP-BOX-I fusion protein yielded the expected increase in p53-dependent transactivation of the p21 promoter relative to the control (FIG. 2C), consistent with previously published data showing that fusion proteins expressing the BOX-I domain sequesters MDM2 protein, thus increasing p53-dependent transcription (Bottger et al., 1997). A titration of each EGFP-peptide construct into both A375 cells and the HCT116 colorectal cancer cell line shows that distinct cell models with a wild-type p53 pathway can give rise to similar changes in the specific activity of p53.

One control performed to address the specificity of the inhibition of the EGFP-phosphopeptide mimetics was to examine cells for changes in the steady-state level of the GFP fusion protein by immunoblotting after transfection (FIG. 2D). It is possible for example, that the higher degree of inhibition by the EGFP-phospho-peptide mimetics is due to higher expression that may non-specifically affect transcription. However, the only major difference observed in the levels of the EGFP fusion proteins were the reduced levels of EGFP-BOX-I protein, presumably due to the ability of the BOX-I peptide-fusion protein to be degraded by the binding of MDM2 (Haupt et al., 1997). These data indicate that the specific activity of each EGFP-peptide fusion protein as an inhibitor or an activator of p53-dependent transcription is correctly reflected in the Relative Light Units used to quantitate p53 activity in FIG. 2C. A second control was set up to examine whether p53-dependent activity could be restored in A375 cells containing inhibitory amounts of the EGFP-phospho-peptide mimetics by co-transfection with increasing amounts of the p300 gene. A dose-dependent increase in p53 activity from the p21 promoter is observed in cells co-transfected with the p300 gene, with the most striking recovery observed using the most potent p53-inhibitor encoded by the-EGFP-S20D construct (FIG. 2E).

The potency of the EGFP-T18D and EGFP-S20D fusion proteins in blocking basal p53-dependent transactivation from the p21 promoter in a cycling cell was a promising observation and it was therefore important to determine if the peptide-fusion proteins would be as effective in blocking p53-dependent transactivation in an irradiated cell. Cells were transiently co-transfected with EGFP-NS, EGFP-BOX-I, EGFP-S15D, EGFP-T18D or EGFP-S20D and p21-Luciferase reporter constructs, followed by treatment with ionizing radiation (5 Gy) or UV-C (20 J/m²). Two hours or five hours after treatment with ionizing radiation or UV-C, respectively, there was an increase in p21-Luciferase reporter activity, indicating that this transient transfection assay can detect radiation-dependent activation of p53 (FIG. 2F). p53-dependent gene expression in the irradiated cells was selectively inhibited by increasing titration the EGFP-phospho-peptide mimetics (FIG. 2F), while the EGFP-BOX-1 peptide stimulated p53 activity in the irradiated cell.

A final model used to examine whether the phospho-peptide mimetic peptides inhibit p53 activity involved the use of the p53-null cell line Saos-2 co-transfected with p300, p53, and the indicated reporter or expression vectors (FIG. 3). Transient co-transfection of increasing amounts of p300 with p53 into Saos-2 cells lead to maximal stimulation of p53-dependent gene expression from the p21 promoter (FIG. 3A) or the bax promoter (FIG. 5A). Using the levels of the p300 and p53 genes that give rise to the highest level transactivation, co-transfection with increasing amounts of the EGFP-S20D peptide on the p21 promoter (FIG. 3B) or the bax promoter (FIG. 5B), inhibits transactivation. As a control, the EGFP-BOX-1 peptide stimulated p53 activity under the same conditions, distinguishing the affects of the BOX-I fusion proteins from the phospho-peptide mimetics.

In Saos-2 cells co-transfected with varying combinations of DNA encoding the EGFP peptide-fusions with p53 and p300, we finally examined the levels of p53 protein and EGFP-peptide fusion protein by immunoblotting (FIG. 3C) in comparison to the p21 reporter activity. The highlights of the data are two-fold. First, the inhibitory EGFP-T18D and EGFP-NS20D peptide fusion proteins were expressed at similar levels relative to the EGFP-NS control in the cotransfected Saos-2 cells (FIG. 3C, middle panel), indicating that the ability of the phosphomimetic peptides to inhibit p53 reflect changes in the specific activity of the aspartate-substituted peptide fusion protein (FIG. 3B). The EGFP-BOX-I peptide fusion protein was expressed at slightly lower levels at lowest point in the titration (1 μg DNA; FIG. 3C), consistent with its lowered expression in A375 cells (FIG. 2D). However, at the higher levels of the EGFP-BOX-1 construct titration (2 μg and 5 μg, FIG. 3C) where p53 activity is stimulated (FIG. 3B), the levels of EGFP fusion were similar to the EGFP-NS controls. The exception in this analysis was the expression level of the EGFP-S15D construct, where missense mutation at position 15 increased expression of the fusion protein relative to all other vectors (FIG. 3C). These latter data indicate that the specific activity of the EGFP-S15D peptide fusion protein as a p53-inhibitor (FIG. 3B) is significantly lower than the other phospho-peptide mimetics. Additionally, these data are consistent with the reduced ability of p300 to bind to the Asp¹⁵ phospho-peptide (FIG. 2A).

The second set of experiments design to compare p53 protein levels under the varying conditions demonstrated that p53 protein levels were increased after titrations of 1 μg of the EGFP-BOX-I, EGFP-S15D, EGFP-T18D, and EGFP-S20D constructs, relative to the EGFP-NS control (FIG. 3C, top panel). This data first suggests that there may be some degree of inhibition of MDM2 binding and subsequent stabilization of p53 protein by all BOX-I variants. However, the induced form of p53 protein is stimulated only using the EGFP-BOX-I construct, as the aspartate substituted fusion proteins presumably bind avidly to p300 and prevent transactivation being triggered by the induced form of p53. Thus, if p53 protein levels in EGFP-transfected cells are plotted as a function of p21-Luciferase activity, then the phospho-peptide mimetics prove to be even higher affinity inhibitors when the data are normalized (data not shown). The set of experiments addressed the correlation between p21-Luciferase reporter activity and endogenous p21 protein expression in the Saos-2 cells (FIG. 2C, bottom panel). Although the luciferase activity is substantially more quantitative, similar qualitative trends can be observed: especially when comparing p21 protein levels in cells transfected with EGFP-NS, EGFP-BOX-I, and EGFP-S20D (FIG. 3C, bottom panel).

Conclusion

Regulation of p53-dependent transactivation occurs by competition for MDM2 or p300 binding to the BOX-I domain of p53 which may be further regulated, in turn, by phosphorylation. It was the purpose of this study to define the role of the recently identified BOX-I phosphorylation sites at Thr¹⁸ and Ser²⁰ as predominantly positive or negative effectors of p300 or MDM2-regulation of p53. Scaffold proteins fused to small peptide regulators have been used in vivo as reagents to dissect regulatory steps in many pathways including cyclin-dependent cdk2 isoforms (Mendelsohn and Brent, 1999; Chen et al., 1999), cdk4 (Ball et al., 1997), p53 (Abarzua et al., 1996), and E2F (Bandara et al., 1997). We report here that scaffold proteins fused to phospho-peptide mimetics of the BOX-I domain of p53 can be used to inhibit p300-coactivation of p53-dependent transcription. These data suggest that the predominant role of the Thr¹⁸ or Ser²⁰ phosphorylation events will be to switch p53 from an MDM2 binding protein to a p300 binding protein, consistent with the cellular models showing that Thr¹⁸ or Ser²⁰ phosphorylation events are associated with activation of p53.

Example of ELISA Test for Screening a Natural Product Library for Their Effect on p53/p300 Binding.

Microtitre wells were coated with 100 ng of streptavidin and incubated overnight at room temperature. To prevent non-specific binding, 100 μl of 3% BSA, PBS, 01% Tween 20 was added and incubated for 1 hour at 4° C. Wells were washed 3 times with 180 μl PBSM 0.1% Tween 20 and biotinylated peptides, resuspended in 50 μl/well of 3% BSA, PBS, 0.1% Tween 20/NaF/Beta-phosphoglycerate, were incubated for 1 hour at 4° C. Non-specific binding sites were blocked by adding 180 μl 5% milk powder, PBS, 0.1 Tween 20/NaF/Beta-phosphoglycerate for 30 minutes at 4° C. before adding p300 resuspended in 50 ml/well of Sf9LB. Where plant extract was added to p300, the reaction was incubated for 30 minutes before adding to wells. The reaction was the incubated for 1 hour at 4° C. and then the wells were rigorously washed 3 times with 180 μl PBS, 0.1%, Tween 20 before incubating for 1 hour with specific antibodies in 50 μl/well 5% milk powder, PBS, 0.1% Tween 20/NaF/Beta-phosphoglycerate. Wells were given another 3 washes with PBS, 0.1%, Tween 20 to remove unbound antibody and specific binding was detected with secondary antibody (anti-mouse or anti-rabbit) conjugated to horse radish peroxidase (HRP). The binding was detected by ECL and quantified using a luminometer (Fluoroskan Ascent FL).

Materials & Methods

Plasmids, Antibodies, & Cells

EGFP-peptides were constructed by ligating double stranded oligonucleotides encoding amino acids 11-30 of human p53 (EGFP-BOXI) and with a codon encoding an aspartate mutant at Ser¹⁵ (EGFP-S15D), Thr¹⁸ (EGFP-T18D or Ser²⁰ (EGFP-S20D), into XhoI/XbaI digested EGFP-C3 plasmid (i.e. EGFP-NS; Clontech). An EGFP-NS control plasmid without an insert was created by ligating XhoI/XbaI ends of EGFP-C3. All EGFP constructs were confirmed by DNA sequencing. The p53-responsive p21-Luc, Bax-Luc and pGL3-Basic constructs were a gift from M. Oren (Weizmann Institute of Science, Israel). pCMV-βGal was a gift from M. G. Luciani (University of Dundee, UK). Full-length p300 and p300(1135-2414) baculovirus were a gift from N. B. La Thangue ((Shikama et al., 1999); University of Glasgow, UK). PCp53-R175H expression plasmid was obtained form Dr Bert Vogelstein (Johns-Hopkins University). pCMVβ-p300 was a gift from M. Giacca (ICGEB, Italy). Full-length MDM2 protein was purified as described (Burch et al., 2000). Full-length p300 and FLAG-p300 (1135-2414) were purified and quatitation determined emperically as described previously (Shikama et al., 1999).

A375 and HCT116 cells both containing a wild-type p53 pathway were maintained in DMEM (Gibco BRL) supplemented with 10% FBS and incubated at 37° C. with an atmosphere of 10% CO₂. For transient transfections, 3×10⁵ cells were plated out in 6-well dishes and transfected with LipofectAMINE (Gibco BRL). The exact quantity of DNA transfected is indicated in each experiment and where necessary, carrier DNA was incorporated to keep the same quantity of DNA consistent in each transfecton. To monitor transfection efficiency, pCMV-βGal was included in each transfection. Unless otherwise stated, cells were harvested 24 hours post-transfection and lysed in Reporter Lysis Buffer and the corresponding luciferase and β-Gal assay carried out according to the manufacturers protocol (Promega, UK).

Immunochemical Assays

Biotinylated peptides were immobilised to streptavidin coated 96-well plates (Dynex Microlite 2) with a titration of 0 B 1 ng/well as indicated previously (Craig et al., 1999b). Essentially, non-reactive sites were blocked in 5% Milk/50 mM NaF/5 mM βPG in PBST20 (0.1% v/v) and emperically-determined levels of p300, FLAG-p300(1135-2414) or full-length Mdm2 proteins were incubated for 1 hour at 4° C. Plates were rigorously washed 3 times with PBST20 (0.1% v/v) to reduce the non-specific binding. Primary antibodies anti-p300(N-15) (Santa Cruz Biotechnology Inc.), anti-FLAG (Sigma) and anti-Mdm2 (2A10) (Movarian Biotechnologies) were used and the appropriate secondary antibody cross-linked to HRP. The signal detected by Enhanced Chemoluminescence was developed using Fluoroskan Ascent FL. Immunoblots were performed for p53 using DO-1 as described previously (Craig et al., 1999a) and antibodies to EGFP were used according to the manufacturers suggestion (Clontech).

### EXAMPLE 2

Results

Developing a Consensus p300-Binding Motif by Mapping its Contact Site in the Ser²⁰ Phosphorylated BOX-I Domain of p53.

The overlapping docking site for MDM2 and p300 on p53 creates a negative and positive effect respectively, on its ability to function as a tumour suppressor protein. Developing peptide-based therapeutics which target these two major effectors would be suitable for signal transduction dissection and subsequent drug development, since both MDM2 (Lundgren, Montes de Oca Luna et al. 1997) and p300 (Ait-Si-Ali, Polesskaya et al. 2000; Kolli, Buchmann et al. 2001) are independently required for cell-cycle progression. The notable differences in the affinity of p300 and MDM2 towards p53-derived phospho-peptides suggested an inherent difference in their binding contacts in the BOX-I domain (Doman and Hupp 2001). To define essential amino acid contacts, a series of alanine-substituted Ser²⁰ phosphorylated peptides were synthesised and the protein-ligand binding ELISA was employed using full-length p300 and full-length MDM2 proteins (FIGS. 6A and B respectively). If Gln¹⁶, Trp²³, Lys²⁴, Leu²⁵ or Leu²⁶ are individually mutated to alanine, then this abrogates the ability of p300 protein to bind to the Ser²⁰ phospho-domain (FIG. 6A). In contrast to p300, a Phe¹⁹, Trp²³ or Leu²⁶ substitution to alanine on the BOX-I derived Ser²⁰ phosphorylated domain inhibits MDM2-binding (FIG. 6B), correlating with the essential residues required for MDM2 protein binding to p53 identified by phage-peptide display (FIG. 7B) and crystal structure of the MDM2-p53 peptide complex (Kussie, Gorina et al. 1996). Interestingly, a search for proteins in the database that have significant homology to the consensus BOX-I p300-binding motif on p53, S^{PO3}XXWKLL (FIG. 6C), identified the HMG Box architectural factor, UBF1 (Upstream Binding Factor 1). UBF necessitates major chromatin remodelling and this S^{PO3} XXWKLL region of UBF is within the domain that is already known to interact with the p300 homologue, CBP (CREB Binding Protein) (Pelletier, Stefanovsky et al. 2000). To determine whether p300 can bind to this region of UBF1 we employed the peptide ELISA containing S^{PO3}XXWKLL consensus peptides from UBF1 (amino acids 231-246 and 321-336) with or without a phosphate moiety at the predicted serine residue. Notably, when a phosphate group was attached to Ser³²⁹ in UBF1 (321-336), which contains a strict homology to the S^{PO3}xxWKLL motif, p300 bound with an affinity slightly less than the p53 BOX-I-Ser²⁰ phospho-peptide (FIG. 6D), suggesting that this may indeed be a consensus contact region for p300. In contrast, UBF1 (231-246) did not bind p300 with or without a phosphate substitution at Ser²³⁹ (FIG. 6D). These differences may be due to the absence of a tryptophan residue at position 242 (FIG. 6C), which is required for p300 binding to the p53 BOX-1-Ser²⁰ phospho-peptide alanine scan (FIGS. 6A and C). These data also suggest that a peptide-based inhibitor derived from the S^{PO3}xxWKLL motif could be generated to specifically target the p300-p53 or UBF-CBP axis. Since our interests lie primarily within the p53-p300 axis, we intended to identify the phosphate binding domains on p300 that bind to the Ser²⁰ phospho-domain of p53.

Identification of Phospho-Serine20 Binding Domain-1/2 (POD-1/2) on p300

Various techniques have been utilised to define the p53-p300 binding sites, most having employed the use of GST pull-down assays with GST-p53 fusion proteins and p300 mini-proteins. From our previous studies, we demonstrated that an EGFP-S20D phospho-peptide mimetic was able to function like the BOX-1 Ser²⁰ phospho-peptide by binding to p300 in vitro and in inhibiting p53-dependent transcription in cells (Dornan and Hupp 2001). Transcriptional inhibition by the EGFP-S20D fusion protein (quantitated by RLU's driven by luciferase activity from a p21 promoter) was recovered with ectopically expressed full-length p300 (FIGS. 6E and 11C), indicating that p300 is indeed one of the targets for EGFP-S20D protein. This transcriptional-recovery assay was used to map the EGFP-S20D peptide binding region on p300 by transfecting p300 mutants spanning the entire protein and determining which mini-p300 domain could recover transcriptional inhibition.

Inhibitory levels of EGFP-S20D (FIG. 6E) were co-transfected with the p21-Luciferase reporter and GAL4, GAL4-p300, GAL4-p300 (1-504), GAL4-p300 (1-703), GAL4-p300 (192-504), GAL4-p300 (192-600), GAL4-p300 (192-703), GAL4-p300 (192-1004), GAL4-p300 (504-1238), GAL4-p300 (852-1071), GAL4-p300 (636-2414), GAL4-p300 (1064-2414), or GAL4-p300 (1757-2414). Transfection of the full-length p300 protein recovers the transcription inhibition induced by the EGFP-S20D, relative to EGFP-NS and GAL4 controls for basal transactivation on the p21 promoter. Interestingly, the EGFP-S20D transcriptional inhibition was recovered and enhanced by constructs encompassing the N- and C-terminus of p300. The constructs that did not recover transcription inhibition were from amino acids 504-1238 which contains the CRD1 (p21-inducible transcriptional repression domain) (Snowden, Anderson et al. 2000), the bromodomain and KIX (KID Interaction) domain (a previously published p53 interaction site (Van Orden, Giebler et al. 1999)), and amino acids 852-1071 which incorporates the CRD1 domain only.

Since relatively large domains in the N- and C-terminus of p300 recovered transcription inhibition induced by the EGFP-S20D peptide, fine-mapping of the peptide-binding sites was performed using a second set of p300-mini proteins which included: GAL4-N1 (aa 2-337), GAL4-N2 (aa 302-667), GAL4-N3 (aa 407-566), GAL4-C1 (aa 1737-2414), GAL4-C2 (aa 1945-2414) and GAL4-C3 (aa 1709-1913). Inhibition by EGFP-S20D was recovered by GAL4-N2 and GAL4-N3, but not GAL4-N1, indicating that in this assay the EGFP-S20D peptide has an affinity for amino acids 407-566, POD-1 (FIGS. 6F and 6H). GAL4-C1 and GAL4-C2, but not GAL4-C3, were able to recover transcription inhibition by EGFP-S20D suggesting that the C-terminal docking site for the EGFP-S20D peptide lies between amino acids 1945-2414, POD-2 (FIGS. 6F and 6H). The N-terminus of p300 has previously been shown to interact with a host of transcription factors including p53, HIF-1 and Stat-2. However, few have been mapped outside the C/H1 (TAZ1) domain, but include Stat-1, SF-1 and NHR. The POD-1/2 domains of p300 fall into the latter class and define novel functional motifs whereby p300 interacts with p53.

To determine whether the in vivo transcriptional recovery data reflect an intrinsic specificity of the POD-1/2 domains for the BOX-I Ser²⁰ phospho-motif, an in vitro assay was employed to determine whether the POD-1 or POD-2 domains of p300 can bind specifically and directly to the Ser²⁰-phospho-domain (FIG. 6G). Cell lysates obtained from HCT¹¹⁶ (p534%) cells transfected with the indicated GAL4-p300 fusion constructs were incubated with an anti-GAL4 antibody in ELISA wells to capture p300 fragments. The corresponding biotinylated peptide (BOX-I, Ser²⁰-phospho-peptide, or no peptide) was added, and p300-peptide complex stability was quantitated using streptavidin-HRP coupled to luminography. This experiment yielded data that mirrored the in vivo peptide-recovery data with the EGFP-S20D construct, where the p300 fragments containing the 17 kDa POD-1 mini-protein (GAL4-N3) or the 45 kDa POD-2 mini-protein (GAL4-C2) display specificity for the BOX-I Ser²⁰-phospho motif of p53, relative to the negative control GAL4 alone or the positive control GAL4-full length p300 (FIG. 6G). Identification of novel p300 binding motifs by phage-peptide display

Reconstitution of the p300-p53 complex has thus indicated that two phosphate-binding domains in p300 play a role in contacting the BOX-I Ser²⁰-phospho-domain of p53 (FIG. 6H) and that the consensus derived from the p300-binding reaction is distinct from that observed for MDM2 (FIGS. 6A and B). Given the multiple roles of the p300/CBP transcriptional co-activators in cell growth, transformation and development (Goodman and Smolik 2000), the number of potential binding partners is staggering. These co-activators are thought to be present at limiting levels within the cell (Horwitz, Jackson et al. 1996) thereby allowing the propensity for cross-talk between transcription factors competing for the same pool of co-activators (Webster and Perkins 1999). However, it is possible that the nucleus assigns compartments to increase the local concentration of select co-activators and transcriptional machinery for a specific transcriptional program (Lemon and Tjian 2000). In order to determine whether other p53 binding sites are targeted by p300, we utilised phage-peptide display as an approach to obtain novel peptide docking sites for p300 and search the enriched p300-binding peptides for homology to motifs in p53.

The full-length p300 used as bait in phage-peptide display was biochemically active as defined by the BOX-I Ser²⁰-phospho-domain binding assay (FIG. 6A). The use of a 12-mer phage-peptide library with native p300 as the target protein, yielded the generation of a PXP, PXXP and PXXXP (where P is Proline and X is any amino acid) motif after three rounds of selection (FIG. 7A). It is interesting that we did not obtain basic polypeptides derived from p53 or histones that are targeted by the acetyltransferase activity of p300, which suggest these peptides may display a relatively low affinity for p300. The proline-rich motifs that were bound by p300 are actually present in a variety of transcription factors that are already known to recruit co-activators which mediate gene expression (FIG. 7C; e.g. p53, ROR2α, Smad4 and NKX2.5) ((Grossman, Perez et al. 1998; Lau, Bailey et al. 1999; de Caestecker, Yahata et al. 2000) (Poizat, Sartorelli et al. 2000)) and are present in proteins not known to bind to p300 (data not shown).

A control was performed to measure the integrity of the phage-peptide library to ensure that the polyproline peptide selection was specific for the target protein p300 (FIG. 7B). Since MDM2 binds to distinct residues within the same region of p53 (FIGS. 6A and B) to which p300 binds, this provided us with a good control and MDM2 was therefore used as bait for the phage-peptide selection. Peptides were identified yielding the expected residues within the BOX-I domain of p53 required for MDM2 binding (FIG. 6B and FIG. 7B; Phe¹⁹, Trp²³ and Leu²⁶). Since p300 and MDM2 both target overlapping domains on p53, it was surprising that by phage-peptide display, a p300 binding-peptide with homology to the BOX-I domain was not selected. However, phosphorylation of the BOX-I domain is essential for p300 binding to peptides from this motif ((Dornan and Hupp 2001) and FIG. 6D).

To verify that p300 can bind to the predicted sequences from the database search for polyproline motifs (FIG. 7C), we selected peptides containing sequences spanning the transcription factor Smad-4 proline-rich region (aa 275-303) and the p53 polyproline region (aa 64-92) and tested their binding affinity by ELISA (FIG. 7D). The C-terminal end of the SMAD4 polyproline domain bound with a relatively high affinity to p300 compared to the N-terminal proline-rich domain (FIG. 7D). Deletion of the SMAD4 polyproline domain of SMAD-4 abrogates its activity as a transcription factor (de Caestecker, Yahata et al. 2000), but the mechanism for this loss of activity was not defined. Our data suggest that the reason for loss of SMAD4 activity may be due to reduction in p300-SMAD-4 complex stability. As predicted by phage display, p300 bound to two of the three p53 polyproline peptides containing amino acids 55-74 and 83-102 (FIG. 7D) with high affinity, suggesting that this is indeed a potential binding site for p300 that may modulate p53's function as a tumour suppressor. Phosphorylation of p53 at Thr⁸¹ was reported to stimulate p53-dependent transcription (Buschmann, Potapova et al. 2001), however the addition of a phosphate to Thr⁸¹ did not restore binding activity of p53PRO (69-88) to p300 (data not shown).

Acetylation of p53 by p300 is Stimulated by DNA

The polyproline domain is required for p53-dependent transcription from some promoters, but a direct mechanism for this phenomenon has not been defined (Venot, Maratrat et al. 1998; Zhu, Jiang et al. 1999; Baptiste, Friedlander et al. 2002). Given the direct binding of p300 to the polyproline region of p53 (FIG. 7D), this region may regulate p53-dependent transcription by direct recruitment of p300. We designed in vitro assays to measure the effect of the polyproline domain of p53 on: (1) the stability of the p300-p53 complex in the presence or absence of DNA; (2) the stability of the p300-p53 complex in the absence or presence of acetyl-CoA; and (3) p300 acetylation of p53 and histones in the absence or presence of DNA. The acetyltransferase activity of p300 and its homologue CBP on histone and non-histone substrates has been well documented (Goodman and Smolik 2000; Vo and Goodman 2001). The role for acetylation on the N-terminal tails of histones is somewhat more widely appreciated, with the acetylation of lysine residues potentially weakening the nucleosome-DNA interaction, thereby allowing promoter access for the core transcriptional machinery such as TFIID and RNA Pol II. Acetylation of trariscription factors is now a widely observed phenomenon (Zhang and Bieker 1998; Ott, Schnolzer et al. 1999; Sterner and Berger 2000; Masumi and Ozato 2001) but the role and mechanism for this acetylation remains elusive (Prives and Manley 2001). We therefore set out to determine the mechanism behind acetylation of p53 by p300 and establish a role for acetylation in the context of transactivation.

Before setting up an in vitro p53 acetylation assay, the purified p300 protein was first characterised enzymatically using a well-known substrate, histone H4. The kinetics of the acetylation reaction between p300 and histone H4 were determined (FIGS. 8A and B). The data indicate that p300 is behaving like a classical histone acetyl transferase by displaying monophasic kinetics on a histone substrate (Bordoli, Husser et al. 2001) and this preparation of p300 was tested for the ability to acetylate native p53 tetramers expressed in Sf9 cells (Hupp and Lane 1994). Using an antibody against acetylated Lys³⁸² of human p53 and normalising to levels of p53 protein with an anti-p53 antibody, acetyl-CoA-dependent acetylation of p53 was observed, but it was only slightly above background levels and of very low stoichiometry (FIG. 6C; bottom panel, lane 3 vs. lanes 1 and 2). Despite previous observations of strong acetylation signals using peptides as substrates, this suggested two possibilities: (1) phosphorylation at the C-terminus may be blocking acetylation or (2) p53 may need to be in a more favourable conformation for acetylation to occur. Western blotting with phospho-specific antibodies to the C-terminus revealed no phosphorylation was present (data not shown) and thus suggested that p53 may need to be in a more favourable conformation for acetylation to occur. Since p53 most likely interacts with the transcriptional apparatus and co-activators in the context of a eukaryotic promoter, this acetylation reaction was repeated but incorporated a titration of p53 consensus site oligonucleotide, which can induce a conformational change in p53 structure as determined by concealment of monoclonal antibody epitopes and changes in thermostability (Hupp, Meek et al. 1992; Halazonetis, Davis et al. 1993; Hansen, Hupp et al. 1996). Surprisingly, the inclusion of DNA promoted a striking stimulation in p53 acetylation by p300 (FIG. 8D). Since a maximal signal was achieved using 100 ng of oligonucleotides we titrated at a lower range and revealed a does-dependent increase in acetylation of p53 that became maximal at 20 ng (FIG. 8E).

As a control, a titration of DNA into the histone acetylation reaction was performed and DNA did not stimulate or reduce histone acetylation (FIG. 6F) indicating that the DNA-dependence of the p53 acetylation signal observed was p53 specific. The kinetics of p300 acetylation on histones has been extensively studied and displays a ping-pong mechanism, via an ordered reaction involving first the formation of a stable p300acetyl intermediate followed by binding of the histone substrate and transfer of the acetyl moiety to a lysine residue (Thompson, Kurooka et al. 2001). As a final control to define the integrity of the acetyltransferase reaction, we determined whether the p53-DNA complex acetylation by p300 displays similar kinetics to that of histone substrates. By changing the concentration of acetyl-CoA at a fixed p300 concentration and varying fixed p53 levels, a double reciprocal plot of the initial velocities can be drawn (FIG. 8G). p300 acts in a similar manner to p53-DNA complexes as a substrate compared to histones (Thompson, Kurooka et al. 2001) suggesting that the p53-p300 acetylation reaction similarly displays a ping-pong mechanism as opposed to a sequential (ternary complex) mechanism.

With the p53 acetylation assay biochemically characterised, peptides derived from the polyproline and phosphorylated BOX-I domain of p53 were tested for their ability to inhibit DNA-dependent acetylation of p53, since p300 binds stably to these domains (FIGS. 6 and 7). A titration of BOX-I peptide displayed no effect on the p53 acetylation reaction (FIG. 8H). However, a titration of the BOX-I phospho-Ser²⁰ peptide inhibited p53 acetylation with an IC-50 of approximately 150 μM in this assay. A titration of the polyproline peptide from p53 abrogates the ability of p300 to acetylate p53 with an IC-50 of approximately 75 μM. These data indicate that both the polyproline-binding domain and the phosphate-binding domain of p300 bind at two contiguous sites in the N-terminal domain of p53 to facilitate the DNA-dependent acetylation in the C-terminal domain of p53.

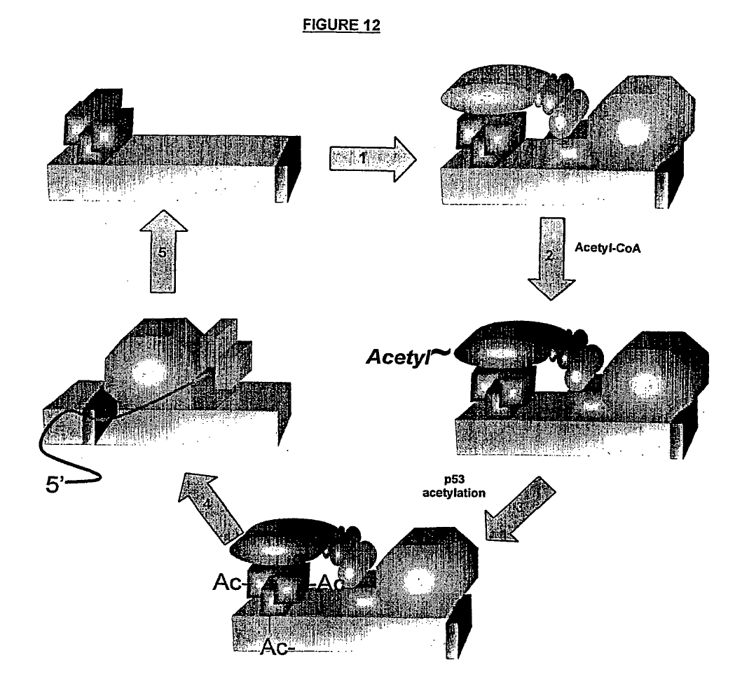

The mechanism of peptide inhibition of p300 acetylation of p53 may be either allosteric, whereby peptide binding changes the conformation of the acetyltransferase domain or the peptides may prevent the docking of p300 on p53. To distinguish between these two possibilities, the same assay conditions were used with histone H4 as a substrate (FIG. 81) and there was no inhibition in histone 14 acetylation using the peptides. This control implies that p300 is docking selectively onto p53 through contacting both the BOX-I domain and the polyproline domain of p53 in order to acetylate the p53-DNA complex, while histone acetylation may simply involve the interaction of the acetyltransferase domain with the lysine tail.

The Polyproline Domain of p53 is Required for p300-p53 Complex Formation, DNA-Dependent Acetylation of p53 Tetramers, and Acetyl-CoA-Dependent De-Stabilisation of the p300-p53 Complex

From the observations using the peptides derived from the polyproline domain of p53 on p300-dependent acetylation of p53, we first determined whether deletion of the polyproline domain on full-length p53 (p53ΔProAE) would reduce acetylation in the C-terminus. Relative to full-length p53 (FIG. 9B, lane 1 and 2), p53ΔProAE was not acetylated by p300 (FIG. 9B, lane 3 and 4) indicating that the polyproline domain is required for this reaction to proceed, which is consistent with the polyproline peptide inhibition data (FIG. 8H).

The acetylation on p53/p53ΔProAE mixed tetramers was next examined since it was possible that interdomain acetylation on the full-length p53 monomer would promote acetylation in the C-terminus of the p53ΔProAE monomer. We expressed p53 and p53ΔProAE in Sf9 cells under conditions where equal amount of p53 mutants and wild-type p53 are known to produce mixed tetramers (FIG. 9A, lane 3; (Bargonetti, Reynisdottir et al. 1992)). The p53/p53ΔProAE mixed tetramers were purified as indicated in Experimental Procedures and subsequently tested for acetylation in reactions catalysed by p300 (FIG. 9B). Notably, the deletion of the polyproline domain on p53 completely abrogates the ability of p300 to acetylate full-length p53 upon the addition of acetyl-CoA relative to the wild-type p53 (FIG. 9B, lanes 5 and 6 vs. lanes 1 and 2). This suggests that docking of p300 on the BOX-I domain on p53 is not sufficient for acetylation by p300 and that the polyproline domain is critical for this post-translational modification to occur.

To further define the mechanism whereby the p53ΔProAE monomer exerts a dominant-negative influence over acetylation of the full-length p53 monomer in the mixed tetramer, we determined whether p300 binding to p53 or p53ΔProAE tetramers was affected by the inclusion of DNA or acetyl-CoA (FIGS. 9C and D). For example, perhaps the inability of p300 to acetylate p53ΔProAE is due to a conformational change caused by deletion of the polyproline domain thereby inhibiting p300 docking. Alternatively, a more stable binding of p53 to p53ΔProAE through the BOX-I domain may promote a dead-end complex incapable of acetylating p53. To distinguish between such possibilities, p300-p53 binding was quantified in the absence or presence of DNA (FIG. 9C) and in the absence or presence of acetyl-CoA (FIG. 9D).

p300-p53 complexes were stabilised by adding increasing amounts of DNA (FIG. 9C, panel 1), which is consistent with a previous report that p300-p53-DNA complexes can be detected using DNA-binding band-shift assays (Lill, Grossman et al. 1997). However, two features were evident from quantitating p300-p53ΔProAE complex stability. First, in the absence of DNA there was a reduced, but still significant level of p300-p53ΔProAE complex formed (FIG. 9C, panel 3). Second, the inclusion of increasing amounts of DNA abolished p300-p53ΔProAE complex formation (FIG. 9C, panel 3). These data indicate firstly that the polyproline domain is required for efficient binding by p300 and is consistent with the ability of p300 to bind stably to polyproline containing peptides derived from p53 or SMAD-4 (FIG. 7D). Secondly, the dissociation of the p300-p53ΔProAE complex by DNA may explain why there is no detectable acetylation of this substrate. Consistent with this, the mixed p53/p53ΔProAE tetramer also binds more weakly to p300 (FIG. 9C, panel 5) and this complex is also de-stabilised by including increasing amounts of DNA in a dose-dependent manner (FIG. 9C, panel 5). The de-stabilisation of p300-p53ΔProAE complex by DNA explains the inability of the mixed tetramer to be acetylated by p300 in the presence of DNA (FIG. 9B).

Paradoxically, acetylation can serve as a positive signal for CBP and TRAP binding in a ChIP assay (Barlev, Liu et al. 2001) or a negative signal by recruiting histone deacetylases (HDACs) such as hSir2 (Luo, Nikolaev et al. 2001) but the actual effect on the interaction between p53 and p300 remains unknown. With the role for acetylation somewhat elusive at present, we wished to determine if acetylation had a polyproline-dependent effect on the association or dissociation of the p53-p300 complex. To address this issue we quantified the binding of p300 to p53 in an ELISA before and after the addition of acetyl-CoA in an acetylation reaction utilising the p53ΔProAE and p53/p53ΔProAE as controls (FIG. 9D). These data show that there is a remarkable decrease in p300 binding to p53 after the inclusion of acetyl-CoA (FIG. 9D, panel 3 vs. panel 6) when p53 becomes acetylated (FIG. 9D, panel 2 vs. panel 5), implying that the acetylation of p53 is promoting the dissociation of p300 from the DNA-p53-p300 complex. The binding of p300 to the p53ΔProAE (FIG. 9D, panel 3) and p53/p53ΔProAE (FIG. 9D, panel 3) is not destabilised by the addition of acetyl-CoA (FIG. 9D, panel 3 vs. panel 6). No acetylation of the polyproline-deleted p53 tetramers was detected using this methodology (FIG. 9D, panel 2 vs. panel 5) confirming the previous observations (FIG. 9B). Together, these molecular data indicate that the polyproline domain of p53 is required for; (1) p300:p53 complex formation; (2) p53 acetylation; and (3) de-stabilisation of the p300:p53 complex by acetylation. Given the recent evidence that acetylation may not activate the latent DNA binding activity of p53 and that the C-terminus of p53 is a positive regulatory domain with respect to transcription (Espinosa and Emerson 2001), these data also suggest that the role of acetylation may be to promote the turnover of the p300-p53 complex (FIG. 12D).

p53-Dependent Transactivation is Compromised by Deletion of the Polyproline Domain

To confirm the relevance of the polyproline domain to p53 function in vivo, cell-based assays were developed to determine whether (1) the polyproline domain is required for p53-depenent transcription; (2) the site for polyproline contact (SPC) on p300 mapped to a distinct region from the POD-1/2 domains; and (3) the POD-1/2 and SPC domains play a role in p300-dependent cell-cycle progression.

The transcription activity of p53 deleted in the polyproline motif has been the focus of previous studies, but no direct biochemical mechanism has been identified for (1) the reduced transactivation activity on some promoters (Zhu, Jiang et al. 1999); (2) the modulation of proteosome-mediated degradation (Berger, Vogt Sionov et al. 2001; Buschmann, Potapova et al. 2001); or (3) the apoptotically compromised phenotype of cells expressing this mutant form of p53 (Walker and Levine 1996; Sakamuro, Sabbatini et al. 1997). Since our biochemical evidence suggests a direct role for the polyproline domain in mediating p53-p300 complex interaction, we investigated the activity of p53 lacking the polyproline domain (p53ΔProAE) on its ability to transactivate and synergise with the co-activators p300 and hCBP on either p21 or bax Luciferase-reporter constructs or on endogenous p21 and bax promoters. To address this issue, Saos-2 (p53−/−) cells were co-transfected with p53 or p53ΔProAE along with either p300 or hCBP and the corresponding Luciferase-reporter constructs (p21 or bax) and transactivation activity was measured by relative luciferase activity. p53-dependent transactivation of the p21 and bax promoters was hindered by deletion of the polyproline domain of p53 (FIGS. 9E and 9F, lane 4 vs. lane 5) despite expression levels of the wild-type and mutant proteins being similar. More strikingly, the p53ΔProAE mutant's ability to synergise with p300 or hCBP was abrogated on the p21 and Bax promoters (FIGS. 9E and 9F, lanes 6 vs. 7). These results suggest the need for the polyproline domain to promote p53-dependent transcription from genes whose products mediate growth arrest or apoptosis. Since the nature of this transcription assay depends on reporter constructs, which have not necessarily been processed with eukaryotic chromatin assembly proteins and subsequent chromatin remodelling enzymes, the response from the endogenous p21 and bax promoters was normalised by western blotting of endogenous proteins (FIGS. 9E and 9F, respectively). Induction of p21 and Bax proteins was severely reduced after transfection of the p53ΔProAE mutant, relative to wild-type p53 (FIGS. 9E and 9F, lanes 4 vs. 5). When normalised to total p53 protein or p53ΔProAE protein in the absence or presence of transfected p300, both p21 and Bax proteins exhibit reduced levels of induction using the p53ΔProAE mutant (FIGS. 9E and 9F, lanes 6 vs. 7). The results obtained from the transiently transfected reporter constructs mirrored those of the endogenously expressed proteins, suggesting that the p53ΔProAE mutants inability to efficiently transactivate the p21 and bax promoters is not due to differences in chromatin arrangement or location.

Negative regulators as well as positive effectors like p300 can in theory target the polyproline motif. For example, the ubiquitin ligase NEDD4 is a WW domain-containing protein and we have evidence that this inhibits p53 activity in transient transfection assays through its binding to the polyproline domain of p53 (data not shown). However, p300 binding seems to be the dominant function for the polyproline domain in vivo because if the binding and inhibition of p53 by NEDD4 were the major function of this polyproline motif, then the p53ΔProAE mutant should have more activity in transfection assays. In addition, the p53ΔProAE mutant has enhanced polyubiquitination and decreased half-life due to enhanced binding by MDM2 protein (Buschmann, Potapova et al. 2001). Presumably the enhanced stability and reduced ubiquitination of full-length p53 containing the polyproline domain cannot be explained by enhanced NEDD4 binding via the WW domain, but through enhanced p300-p53 binding. p300 has been shown to be required for p53 protein stabilisation after DNA damage (Yuan, Huang et al. 1999). Since the polyproline domain of p53 appears to function as a positive scaffold-binding site to stimulate p53 activity in vivo, we continued to focus on the role of p300 in docking to the polyproline domain of p53.

Polyproline Peptide-EGFP Fusion Protein Selectively Inhibits Endogenous p53-Dependent Transcription In Vivo

Small-peptide ligands, derived from specific transcription factors that bind to p300/CBP, may give rise to leads for assays designed to acquire promoter specific transcription inhibitors (Kung, Wang et al. 2000; Dornan and Hupp 2001). An EGFP-fusion polyproline-peptide (EGFP-PRO) was generated to determine if such a bioactive peptide would harbour the ability to inhibit endogenous p53-dependent transcription. As controls, a non-specific peptide (EGFP-NS), a p53 transcription stimulator (EGFP-BOX-I), and a p53 inhibitor (EGFP-S20D) were also transiently co-transfected into cycling A375 cells with a p21-Luciferase reporter vector and a control pCMV-β-Gal. The EGFP-PRO peptide harboured the ability to inhibit endogenous p53-dependent transcription compared to the EGFP-NS peptide and with the same potency as the EGFP-S20D peptide (FIGS. 10A and 10B). The ability of the EGFP-PRO peptide to inhibit p53-dependent transcription suggests that the polyproline domain is indeed required to mediate p53-dependent transcription from the p21 promoter. An immunoblot with the transfected lysates carried out with a specific antibody to EGFP demonstrated that the fusion-proteins were expressed at similar levels (FIG. 10B). The reduced steady-state levels of the p53-transcription stimulator EGFP-BOX-I compared to controls (FIG. 10B) was reported previously (Dornan and Hupp 2001) and is presumably due to the ability of MDM2 to bind to the BOX-I domain and promote the degradation of heterologous fusion proteins (Dumaz and Meek 2000).

There are other potential factors that bind to the polyproline domain of p53 that may be the actual target of the EGFP-PRO inhibitor, which include JNK (Buschmann, Potapova et al. 2001), WOX-1 (Chang, Pratt et al. 2001), CSN (Bech-Otschir, Kraft et al. 2001) and mSin3a (Zilfou, Hoffman et al. 2001). However, a titration of ectopically expressed p300 into cells containing inhibitory levels of the EGFP-PRO peptide was able to recover p53-dependent transcription (FIG. 10C). These data suggest that the polyproline peptide is indeed inhibiting p53-dependent transcription by virtue of binding to p300.

To examine whether the polyproline peptide can inhibit p53-p300 co-stimulation of transcription, Saos-2 (p53−/−) cells, were co-transfected with p53 and p300 at levels where synergy is evident with the p21 and bax-Luciferase promoters (FIGS. 10D and 10E). Following a titration of EGFP-NS, EGFP-BOX-I, EGFP-S20D or EGFP-PRO peptide (FIGS. 10F and 10G), there was a striking inhibition of p53-p300 co-stimulation on the p21 and bax promoters in a dose-dependent manner after expression of the EGFP-PRO peptide. As expected, the EGFP-BOX-I peptide stimulated p53 activity and EGFP-S20D inhibited synergism between p53 and p300 (Dornan and Hupp 2001), indicating the polyproline domain appears to be involved in the p300-transactivation process rather than the MDM2-dependent inhibition pathway. The importance of the polyproline domain is highlighted by the fact that almost 200 tumours harbour mutations within this region of p53 (Buschmann, Potapova et al. 2001), thereby presumably abrogating the ability of p53 to function as a genetically programmed transcription factor. We therefore went on to map the site for polyproline contact (SPC) on p300.

Identification of the Site for Polyproline Contact (SPC-1/2 Domain) on p300

We determined whether the EGFP-S20D and EGFP-PRO fusion proteins bound to identical or dissimilar regions on p300 since they both appear to modulate p53-dependent transcription. Transcriptional inhibition by EGFP-PRO fusion proteins was recovered with ectopically expressed full-length p300 (FIG. 11A), indicating that p300 is indeed one of the targets for EGFP-S20D (FIG. 6E) and EGFP-PRO fusion proteins. This transcriptional-recovery assay was used to identify the EGFP-PRO peptide-binding region on p300. Inhibitory levels of EGFP-PRO (FIG. 10B) were co-transfected with the p21-Luciferase reporter and GAL4-p300 mini-proteins as described previously for mapping the EGFP-S20D peptide binding region of p300 (FIG. 6E).

Mapping of the polyproline docking-site on p300 was performed using the in vivo peptide-inhibition recovery assay used for mapping the POD-1/2 domains (FIGS. 6E, 6F, and 6G) in order to determine whether the polyproline-binding domain is proximal or is contained within the POD-1/2 domains. Using large fragments of p300 (FIG. 11A), the SPC domains interestingly also map to one region in the N-terminus and a second region in the C-terminus of p300 (FIG. 11A), which are both identical to the position of the POD-1 and POD-2 domains of p300 (FIG. 11A vs. FIG. 6E). However, fine mapping revealed significant differences in the ability of the N-terminal and C-terminal p300 mini-proteins to recover transcriptional inhibition by EGFP-S20D and EGFP-PRO peptides (FIGS. 6F and 11B respectively). EGFP-PRO mediated inhibition was recovered by GAL4-N1, GAL4-C1 and GAL4-C3, but not GAL4-N2, GAL4-N3 or GAL4-C2. Thus, the SPC-1 domain flanks the POD-1 domain in the N-terminus of p300 and the SPC-2 domain flanks the POD-2 domain in the C-terminus of p300 (FIG. 11D).

An in vitro assay was employed using the GAL4-p300 mini-proteins in order to determine whether evidence could be found for a direct binding between the minimal SPC-1 and SPC-2 domains and the polyproline peptide (FIG. 1C). Cell lysates obtained from HCT116 (p53^{-/-}) cells transfected with the indicated GAL4-p300 fusion constructs were incubated with an anti-GAL4 antibody in ELISA wells to capture p300 fragments, the corresponding biotinylated peptide (BOX-I, polyproline peptide, or No peptide) was added, and p300-peptide complex stability was quantitated using streptavidin-HRP coupled to luminography. As utilised for the EGFP-S20D (FIG. 6G), it was confirmed that the 40 kDa SPC-1 mini-protein (GAL4-N1) and the 21 kDa SPC-2 mini-protein (GAL4-C3) bind selectively to the polyproline peptide derived from p53 (FIG. 11C). These data indicate that the N-terminal docking site for the polyproline peptide is between amino acids 192-302, SPC-1 and the C-terminal docking site is between amino acids 1709-1913, SPC-2 (FIG. 11C). In summary, these molecular studies show that two contiguous domains on p300 (SPC-1 and POD-1+SPC-2 and POD-2) are required to assemble p300 onto two contiguous regions on p53 harbouring a phospho-motif and a polyproline motif.

EGFP-PRO Transcription Inhibitors Selectively Induce a G2/M Arrest in Cycling Cells Independent of the p53 Status

p300 is required for stabilising p53 protein after DNA damage (Yuan, Huang et al. 1999) and inducing p53-dependent growth arrest or apoptosis (Yuan, Huang et al. 1999), but it is also required more fundamentally for cell-cycle progression (Lee, Sorensen et al. 1998). Thus, p300 may serve as an attractive target for the design of agents that can modulate the rate of cell-cycle progression independent of p53. The p53 transcription inhibitory peptides (EGFP-S20D and EGFP-PRO) were transfected into cycling HCT116 p53^{+/+} and p53^{-/-} cells to determine whether agents that bind to the POD-1/2 domain of p300 or the SPC-1/2 domain of p300 affect cell-cycle progression in the absence or presence of p53.

To address this issue, we co-transfected EGFP-NS, EGFP-BOXI, EGFP-S20D or EGFP-PRO with pCMV-CD20 cell surface marker into HCT116 (p53^{+/+}) cells, selected the transfected population using a FITC-conjugated CD20 antibody, sorted them by FACS and the resultant cell cycle profile of the transfected population determined by propidium iodide staining of DNA. The amount of EGFP-peptide construct and levels of EGFP protein produced were identical to those used in transcription assays (FIG. 10 and data not shown), so a direct correlation can be made between the reduction in transcription by the EGFP-S20D or EGFP-PRO peptides and changes in cell-cycle parameters. Transfection of EGFP-S20D peptide induced a prominent G2/M arrest when compared to EGFP-NS control (FIG. 12A and 12C) suggesting that the POD-1/2 domains are required for cell-cycle progression at the G2/M boundary. Interestingly, the EGFP-PRO peptide revealed a more striking arrest in G2/M phase suggesting that this may be a more potent inhibitor of p300 complex association and that the SPC-1/2 domains have a more significant role than the POD-1/2 domains in cell cycle progression. By contrast, the EGFP-BOX-I fusion protein, which can stimulate p53-dependent transcription (FIG. 9), surprisingly had a relatively insignificant role in altering cell-cycle parameters. The MDM2-binding peptides have been well-documented to have a positive affect on p53^{-/-} dependent transcription (Bottger, Bottger et al. 1996). However, very little has been done with regard to the activity of MDM2-binding ligands as potential therapeutics and this data suggests that MDM2 inhibition in p53^{+/+} cells may not have significant pharmacological affects.

With the arrest in G2/M observed in p53^{+/+} cells using the PRO and S20D fusion peptides, it was determined whether the peptides would have any effect on the cell cycle profile in a p53^{-/-} cell line. Using the same parameters as in the p53^{+/+} cell line, the effects on the cell cycle were determined (FIGS. 12B and 12D). The EGFP-PRO peptide similarly induced a G2/M arrest in the p53^{-/-} cells (FIGS. 12A and 12C) suggesting a role for the SPC-1/2 domains of p300 in cell cycle progression independent of p53. In contrast, it is notable that the EGFP-S20D peptide failed to elicit a defined G2/M arrest indicating that there are striking differences between the functions of the SPC-1/2 and POD-1/2 domains on p300 in cell-cycle control. First, the POD-1/2 domains can be targeted as potential therapeutics only in the presence of p53, suggesting a role for the phosphate-binding domain of p300 and kinases that target these motifs like CHK2 (Chehab, Malikzay et al. 2000; Hirao, Kong et al. 2000) as modifiers of the p53 pathway. Second, the more global requirement for the SCP-1/2 domains in cell cycle progression independent of p53 suggest that proline-binding domain and proline containing proteins that are docked by p300 play a more global role in cell-cycle control.

Discussion

The Role of the Polyproline Domain of p53

The oligomeric nature of p53 provides a unique model with which to define conformational elements that modulate the binding and acetylation of a target protein by the transcriptional co-activator p300. The N-terminal BOX-I domain of p53 was shown to contain a p300-binding site, as mutation of this region produces a transcriptionally-inert protein (Lin, Chen et al. 1994). The first evidence for a multi-domain component for the interaction between p53 and p300 came from data showing that phosphorylation of p53 at Ser¹⁵ by DNA-PK stimulates acetylation in the C-terminus (Lambert, Kashanchi et al. 1998). Subsequent studies have shown that phosphorylation of p53 in the BOX-I domain at Thr¹⁸ and Ser²⁰ can stabilise the p300-p53 protein complex (Dornan and Hupp 2001). As different class of p53-activating kinases target the Ser¹⁵, Thr¹⁸, or Ser²⁰ residues (ATM, CK1, and CHK2, respectively), these phosphorylation events provide a method for kinase signalling networks to regulate gene expression by altering the stability of the p300-p53 complex. Developing a consensus phosphate-binding motif for p300 that is distinct from the contact sites involved in the MDM2:p53 complex (FIG. 6) explains why kinase phosphorylation can differentially affect p300-p53 and MDM2-p53 complex stability and led to the identification of UBF as a potential p300 binding protein. During the course of our studies, it was demonstrated that a region of UBF containing the consensus site defined in our work does in fact bind to the p300 homologue CBP (Pelletier, Stefanovsky et al. 2000).

Protein-protein interactions involve a relatively large polypeptide interface that often involves multi-domain docking between polypeptides. In the case of p53, there are a few instances demonstrating the requirement for oligomerisation in heterologous protein-protein complex formation. CHK2 phosphorylation at Ser²⁰ requires and intact tetramerisation domain in the C-terminus of p53 (Shieh, Ahn et al. 2000) while cyclin A-cdk2 phosphorylation of p53 at Ser³¹⁵ requires a cyclin A docking site near the C-terminal acetylation and sumoylation sites (Luciani, Hutchins et al. 2000). Specific DNA binding by p53 itself is modulated by intra/interdomain regulation, for example, where mutation of the CDK2 site can prevent activation of DNA binding by HSP70 binding near the acetylation and sumoylation sites (Hansen, Midgley et al. 1996). Most strikingly, although deletion of the tetramerisation domain of p53 can substantially reduce its activity as a sequence-specific DNA binding protein, a second deletion of the N-terminal transactivation domain can rescue this defect and restore DNA binding, thus providing the most dramatic evidence for intra/interdomain communication between N- and C-terminal domains (Jayaraman, Freulich et al. 1997).

p300 binding to p53 is likely to involve a complex association reaction given the fact that structure/function studies have shown that oligomerisation influences p53 binding to regulatory proteins. Therefore, phage-peptide display was used as a method to acquire high-affinity peptide ligands that could define novel protein-protein contacts that may be important in p300-protein docking to p53. One set of such enriched peptides contained polyproline repeats (FIG. 7) and displayed homology to proline-rich regions of transcription factors that are known to interact with p300 including p53 and SMAD-4. The polyproline domain of p53 was originally shown to differentially modulate growth arrest and apoptotic pathways (Walker and Levine 1996). Peptides derived from the polyproline domain of p53 were shown to activate the latent specific DNA binding activity of p53 in vitro (Muller-Tiemann, Halazonetis et al. 1998), consistent with a positive role for this domain in p53-mediated activities (Walker and Levine 1996), but suggesting that this motif is a negative regulatory domain with respect to sequence-specific DNA binding. Our data showing that the polyproline domain is required for p300-p53-DNA complex formation and DNA-dependent acetylation, rather suggest that the polyproline motif is a positive regulatory domain with respect to p300-dependent function. Subsequent in vivo studies have shown that the polyproline domain regulates the half-life of p53 (Berger, Vogt Sionov et al. 2001; Dumaz, Milne et al. 2001) and that this domain is required for efficient transcription from some promoters. These latter studies have indicated that there are two subdomains for transactivation control in the N-terminus of p53 (BOX-I domain and polyproline domain), but a mechanism whereby the polyproline domain is required for transactivation has not been defined (Baptiste, Friedlander et al. 2002). Our independent identification of p300 as a polyproline binding protein and the requirement for polyproline binding by the SPC-1/2 domains of p300 to stimulate: (1) p300 binding to p53; (2) p300-acetylation of p53; (3) acetyl-CoA dissocation of the p300-p53 complex; and (4) p53-dependent transcription provides a direct mechanism to explain why the polyproline domain can play a positive role in p53-dependent transcription.

The Role of p53 Acetylation: to Acetylate or not to Acetylate?

The reason why p53 is acetylated is somewhat unclear at present (Prives and Manley 2001). Previous studies have suggested a role for acetylation enhancing the DNA-binding activity of p53 (Gu and Roeder 1997; Sakaguchi, Herrera et al. 1998) but more recently it was observed that DNA binding activity was not enhanced with a longer molecule of DNA derived from the p21 promoter (Espinosa and Emerson 2001) compared to short oligonucleotides suggesting that acetylation may not enhance DNA binding activity of p53 at the promoter level. The next potential role proposed by Barlev et al. (Barlev, Liu et al. 2001) is in the recruitment of co-activators p300/CBP and PCAF. Using GST-p53 pull-down assays, a K382R mutant compromised CBP binding to p53 and upon acetylation this interaction was enhanced. However mutation at K319/20/21 to arginine also depleted binding to CBP, suggesting that acetylation at K382 is not sufficient for CBP binding. Despite being a feasible model, some questions still need to be addressed; If acetylation of p53 increases association of co-activators, how does p53 become acetylated since acetyl transferases that target K382 (p300 and CBP) cannot bind in this assay without prior acetylation? Do co-activators need to be present in a multimeric complex for binding to Ac-p53?

The most surprising biochemical observation from these studies was both the striking DNA-dependence of p53 acetylation and the strict requirement for the polyproline domain of p53 to facilitate acetylation of the DNA-bound p53 tetramer by p300. Previous studies on histone acetylation have shown that the reaction proceeds through a ping-pong mechanism whereby a p300˜acetyl intermediate precedes histone acetylation and that long regions of DNA can inhibit acetylation. There has been little information acquired on the mechanism of acetylation on an oligomeric histone octamer using full-length p300 and most kinetic studies have been performed using small basic peptides as substrates for the acetyltransferase reaction. The DNA-dependence in p53 acetylation suggests that the C-terminus of p53 is cryptic with respect to the p300 acetyltransferase domain and that a conformational change in the p53 oligomer is required to allow the C-terminus accessibility to the active site of p300. The mechanism whereby the polyproline domain of p53 facilitates p300-dependent acetylation was identified by characterising DNA-dependent acetylation using p53ΔPro. This mutant p53 bound to p300 with a lower affinity than full-length p53, however, DNA completely destabilised the p300:p53ΔPro complex, thus indicating why p300 cannot acetylate p53ΔPro in the presence of DNA, since wild-type p53 and p53ΔPro can bind oligos derived from the p21 and bax promoter with equal affinity (Venot, Maratrat et al. 1998). Further, acetyl-CoA cannot de-stabilise the p300:p53ΔPro complex as well as that observed using wild-type p53 suggesting that acetylation and not acetyl-CoA binding is required to turnover the p300:p53 complex. Future work will involve understanding the detailed mechanism of how p300-docking can promote acetylation of a substrate. For example, whether intra/interdomain docking onto the tetrameric p53-DNA complex facilitates acetylation of a specific monomer or whether the reaction is less stereo-specific will require further reconstitution of mixed tetramers containing various deletions or point mutations in specific motifs. Whether other oligomeric substrates such as histone octamers harbour binding determinants other than the basic residues will require reconstitution of the histone octamer and further biochemical studies. Additionally, whether other transcription factors that bind DNA like SMAD-4 or transcription regulators that do not bind DNA such as Rb similarly have p300-docking sites that are distinct from the acetylation site and whether acetylation of these substrates are modulated by DNA or protein co-factor binding remains to be determined.

Another potential model that has been proposed for the role of p53 acetylation is in the recruitment of transcriptional repressors such as hSir2 (Luo, Nikolaev et al. 2001) which is indeed a potentially novel role for acetylation in the context of p53 regulation. With the previous observations that mSin3a also binds the polyproline domain of p53 (Murphy, Ahn et al. 1999; Zilfou, Hoffman et al. 2001) this could propose a model whereby p300 binds the polyproline domain and acetylates p53 and thereby acts as a signal for mSin3a to recruit HDACs to switch off transcription or there may be competition between transcriptional co-activators and repressors for their substrate at the promoter level. Our observations suggest that acetylation promotes the dissociation of p300 or p53 from p53-p300 complexes (FIG. 9D) which may tie in with the recruitment of repressors to p53 (Murphy, Ahn et al. 1999, Luo, Nikolaev et al. 2001; Zilfou, Hoffman et al. 2001) promoters as a mechanism of turning off transcription. On the other hand, this may suggest that the rapid dissociation of p53-p300 complexes from the promoter and the concurrent increase in p53 levels and p53 acetylation observed after DNA damage (Sakaguchi, Herrera et al. 1998) facilitate promoter clearance to allow the rapid synthesis of mRNA and the re-initiation complex formation.

However tempting these models may seem, they need to be placed in a context to aid in the understanding of p53 regulation and function. For example, it is well known that p53 can transactivate and repress many target genes (Wang, Wu et al. 2001) (Wang, Wu et al. 2001) and the mechanism behind each of these is still largely unknown. In the context of transactivation, p300 may bind the BOX-I and polyproline motifs of p53 via its SPC-1/2 and POD-1/2 domains thereby communicating with a pre-initiation complex, bind acetyl-CoA and form the high energy p300-acetyl complex, acetylate p53, this in turn may promote the dissociation of p300 or p53 from p53-p300 complexes and thereby aid in the required promoter clearance stage for efficient synthesis of nascent mRNA and transcriptional re-initiation (FIG. 12). In the context of transrepression, p300 may be recruited to acetylate p53 at the promoter and promote the association with repressors or via a p300/acetylation-independent pathway. Since chromatin arrangement plays a major role in regulating gene expression, it is possible that specific areas of the nucleus contain individual compartments that have local concentrations of p53/p300 and p53/mSin3a to engage in a specific transcriptional program at a specific local chromatin area that in itself may be regulated by cell cycle progression and/or stress responses dictated by structural changes in the nuclear matrix (Lemon and Tjian 2000).

The Role of the Polyproline-Binding Domain of p300

The role of the transcriptional co-activator p300 in mediating p53-dependent transcription and the mechanism behind this regulation has only begun to be elucidated. The number of p300 binding partners is increasing and elucidation of the mechanisms of these interactions is at an early stage (Goodman and Smolik 2000; Vo and Goodman 2001). However, the versatility of p300 establishes a gateway into the therapeutic intervention by small ligands. For example, the use of p300-truncated fragments in rat mesangial cells has suggested that the N-terminus of p300 is important for growth arrest function and the C-terminus modulates apoptosis (Segelmark, Barrett et al. 2000). Thus, the concerted arrangement and phosphorylation status of p53 tetramers and p300 placement on the promoter may dictate the efficiency of gene transactivation since the S20D and PRO domains of p53 bind to distinct sites in both the N-terminus and C-terminus of p300. Identifying the interaction sites on transcription factors could lead to the generation of small molecular weight ligands programmed to intervene with a specific transcription program and ultimately modify aberrant signals within the cellular transcription machinery (Kung, Wang et al. 2000).

Previous studies on mapping the p53-p300 interactions have focussed on the use of GST fused N- and C-terminal fragments of p53 in pull-down and immunoprecipitation experiments. For example, the C-terminal region of p300 (amino acids 1990-2414) containing the Q-rich domain binds p53 (amino acids 1-72) in vitro, consistent with our findings that p300-GAL4-C2 (amino acids 1945-2414) can recover from EGFP-S20D peptide inhibition of p53 in vivo (FIG. 11C), but distinct from our mapping of the EGFP-PRO peptide binding site to p300-GAL4-C3 (amino acids 1709-1913; FIG. 11C). Other groups have shown interactions between p53 and the KIX domain (Van Orden, Giebler et al. 1999) and C/H1 exclusively, (Grossman, Perez et al. 1998) which are not observed using our peptide-inhibition recovery assay or direct p300-ligand binding assays (FIGS. 10C and 10C). Such differences may be explained by the fact that previous p300-p53 interactions were mapped by protein binding assays, while our assays utilise a combination of p53-dependent transcription and peptide binding to measure p53-p300 co-activation in vivo. Additionally, the presence or absence of co-factors present in crude lysates in GST pull-down experiments may influence p300-p53 complex stability. One other possibility is the role that post-translational modification plays in forming a p300-competent binding form of tetravalent forms of p53. For example, phosphorylation of p53 within the polyproline domain at Thr⁸¹ is associated with stimulating p53 activity, but the mechanism of stimulation was undefined (Buschmann, Potapova et al. 2001). Based on our data, one function of this phosphorylation may be to stabilise the binding of p300 to the polyproline domain. However, the polyproline domain binding to p300 is independent of substrate phosphorylation (data not shown), while phosphorylation of the BOX-1 motif at Ser²⁰ is important for stable binding to p300, suggesting that phosphorylation at Ser²⁰ is more important than at Thr⁸¹ for stabilising the p300-p53 complex. Since most techniques employed to address the binding affinities for various p300 fragments will be non-post-translationally modified, the alternative S20D phospho-peptide binding site that we have identified in our cellular assay may enhance binding of p300 to the polyproline domain (or vice versa) to achieve maximal transactivation activity.

Scaffold protein-peptide display gives unique insight into the specificity of protein-interaction domains of a candidate protein (Brent 2000). Such bioactive peptide-ligands have also been developed as leads for drug-development that target enzymes involved in cell cycle control including Ras-farnesyltransferase, cyclin-dependent protein kinases, and transcription factors such as E2F and HIF-1 (Gibbs and Oliff 1994; Kung, Wang et al. 2000; Morris, Allen et al. 2000). All of these regulatory enzymes impinge on the stress-regulated tumour suppressor p53 pathway and the outcome of drug-responses will depend on the p53 status of a cell. A thorough understanding of the mechanisms controlling p53 transactivation function will undoubtedly lead to the development of a novel range of therapeutics that affect physiologically relevant proliferative or apoptotic pathways modulating diseases ranging from ischemic injury to cancer (Hupp, Lane et al. 2000). The fine mapping of the POD-1/2 and SPC-1/2 domains on p300 suggests that one predominates over the other with respect to p53 activity and cell cycle progression. The polyproline peptide derived from p53 is a more potent inhibitor of DNA-dependent acetylation than the phospho-Ser²⁰ BOX-1 domain peptide (FIG. 81). Although we did not observe significant differences in the ability of the POD-1/2 and SPC-1/2 domains in binding to EGFP-S20D or EGFP-PRO peptides in vivo and compromise p53-dependent transcription (FIG. 10), there were significant differences in how these two peptides affect p300-dependent cell cycle progression. The SPC-1/2 domain appears to play a more significant role in cell cycle progression since the EGFP-PRO correlates with a more potent G2/M arrest In summary, we report on the use of biochemical techniques using active forms of full-length p300 to identify a novel class of peptide-ligand in order to gain new insights into mechanisms of transcriptional regulation. Identification of a polyproline p300-docking site within some transcription factors such as PAX3 and NKX2.5 will provide reagents that can be used for dissecting out developmental pathways under transcription control. We have focussed on one of the polyproline-motif-containing transcription factors, p53, to highlight the significance this motif plays in p53-dependent transcriptional activation. Understanding further how the SPC1/2 and POD1/2 motifs on p300 architecturally assemble onto two distinct domains on the p53 tetramer may assist in designing promoter-specific inhibitors that affect transcription-based genetic programming and growth.

Materials and Methods

Phage-Peptide Display and Biopanning Procedure

A PhD.-12 Phage Display Library Kit (New England Biolabs) based on a combinatorial library of random peptide 12-mers fused to a minor coat protein (pill) of M13 phage was utilised. The displayed peptide 12-mers are expressed at the N-terminus of pill and followed by a short spacer (Gly-Gly-Gly-Ser) and then the wild-type pill sequence. The complexity of the library was 1.9×10⁹ and the titre was 4×10¹² pfu/ml. The biopanning procedure was carried out as previously described (Blaydes, Luciani et al. 2001).

Plasmids & Constructs

EGFP-PRO was constructed by ligating double stranded oligonucleotides encoding amino acids 64-92 of human p53 (EGFP-PRO) into XhoI/XbaI digested EGFP-C3 plasmid (Clontech). EGFP-NS, EGFP-BOX-I, EGFP-S20D, p21-Luc, Bax-Luc, pGL3-Basic and pCMVβ-p300 have been previously described (Dornan and Hupp 2001). pCMV-hCBP was a gift from J. Borrow (Paterson Institute, UK). GAL4, GAL4-p300, GAL4-p300 (1-504), GAL4-p300 (1-703), GAL4-p300 (192-504), GAL4-p300 (192-600), GAL4-p300 (192-703), GAL4-p300 (192-1004), GAL4-p300 (504-1238), GAL4-p300 (852-1071), GAL4-p300 (636-2414), GAL4-p300 (1064-2414), and GAL4-p300 (1757-2414) have been previously described (Snowden, Anderson et al. 2000). GAL4-N1, GAL4-N2, GAL4-N3, GAL4-C1, GAL4-C2 and GAL4-C3 were a gift from Y. Shi (Harvard Medical School, Boston, Mass.).

Cell Culture, Transfections, ELISAs & Western Blots

A375 and Saos-2 cells were maintained in DMEM (Gibco BRL) supplemented with 10% FBS and incubated at 37° C. with an atmosphere of 10% CO₂. Transient transfections and ELISAs were carried out as previously described (Dornan and Hupp 2001). Full-length p300 and His-p300 infected Sf9 cells were harvested 72 hours post-infection as described previously (Shikama, Lee et al. 1999; Dornan and Hupp 2001). Sf9 expressed wtp53 and p53ΔProAE tetramers were purified by heparin-sepharose chromatography as described previously (Hupp and Lane 1994). Transfected lysates were run on a 12% SDS-PAGE and transferred to nitrocellulose membrane and even protein loading confirmed by Ponceau S (Sigma) staining. Primary antibodies anti-p53 (DO-1), anti-p21 (Ab-1) (Calbiochem), anti-Bax (N-20) (Santa Cruz Biotechnology Inc.), anti-EGFP (Clontech) were used and the appropriate secondary antibody conjugated to HRP. The signal detected by Enhanced Chemo-luminescence was developed using autoradiography film (Amersham).

Flow Cytometry

To select the transfected cell population, cells were co-transfected with 3 μg pCMV-CD20 and incubated for 24 hours before washing and detaching cells in PBS/5 mM EDTA. Cells were then harvested and resuspended in serum free media with FITC-conjugated anti-CD20 (Becton Dickinson) and incubating for 30 minutes on ice. Cells were then washed twice with PBS/1% FBS and resuspended in 100 μl PBS before adding 900 μl ice-cold ethanol dropwise. After incubation at 4° C. for >2 hours cells were stained with Pi (40 μg/ml) and treated with RNAse (100 μg/ml). Transfected cells were gated using the FL1 channel and PI staining detected with the FL2 channel and the resultant cell cycle profiles of 2000 cells were analysed using a FACScan and CellQuest software (Becton Dickinson).

Acetylation Reactions

Partially purified p53 (50-400 ng) from Sf9 cells was incubated with 300-400 ng of purified His-p300 in 30-100 μl AT Buffer (50 mM Tris.HCl [pH8], 10% Glycerol, 0.1 mM EDTA, 1 mM DTT, 5 μM TSA and 2 μM Acetyl-CoA.) for 4-10 minutes at 30° C. where the enzymatic reaction was linear. Reactions were incubated on ice for 10 minutes and started with the addition of p300. Acetylation of p53 was detected by the antibody-capture ELISA technique using anti-p53 (ICA9) or by direct western blot using anti-acetyl p53 (AcK373/382) and normalising with anti-p53 (19.1). Histone acetylation was carried out in a similar manner but with the use of 1 μg purified histone H4 (Upstate Biotechnology) as the substrate for p300 and using anti-histone (Roche) and anti-acetyl lysine (Upstate Biotechnology) to detect reaction products. Quantification of acetylation was carried out using bioluminescence (Genegnome).

### REFERENCES

Abarzua, P., LoSardo, J. E., Gubler, M. L., Spathis, R., Lu, Y. A., Felix, A. and Neri, A. (1996) Restoration of the transcription activation function to mutant p53 in human cancer cells. Oncogene, 13, 2477-82.

Ait-Si-Ali, S., A. Polesskaya, et al., (2000). CBP/p300 histone acetyl-transferase activity is important for the G1/S transition. Oncogene 19(20): 2430-7.

Appella, E. and C. W. Anderson (2001). Post-translational modifications and activation of p53 by genotoxic stresses. Eur J Biochem 268(10): 2764-72.

Ashcroft, M., Kubbutat, M. H. and Vousden, K. H. (1999) Regulation of p53 function and stability by phosphorylation. Mol Cell Biol, 19, 1751-8.

Avantaggiati, M. L., V. Ogryzko, et al. (1997). Recruitment of p300/CBP in p53-dependent signal pathways. Cell 89(7): 1175-84.

Ball, K. L., Lain, S., Fahraeus, R., Smythe, C. and Lane, D. P. (1997) Cell-cycle arrest and inhibition of Cdk4 activity by small peptides based on the carboxy-terminal domain of p21WAF1. Curr Biol, 7, 71-80.

Bandara, L. R., Girling, R. and La Thangue, N. B. (1997) Apoptosis induced in mammalian cells by small peptides that functionally antagonize the Rb-regulated E2F transcription factor. Nat Biotechnol, 15, 896-901.

Baptiste, N., P. Friedlander, et al. (2002). The proline-rich domain of p53 is required for cooperation with anti-neoplastic agents to promote apoptosis of tumor cells. Oncogene 21(1): 9-21.

Bargonetti, J., I. Reynisdottir, et al. (1992). Site-specific binding of wild-type p53 to cellular DNA is inhibited by SV40 T antigen and mutant p53. Genes Dev 6(10): 1886-98.

Barlev, N. A., L. Liu, et al. (2001). Acetylation of p53 activates transcription through recruitment of coactivators/histone acetyltransferases. Mol Cell 8(6): 1243-54.

Bech-Otschir, D., R. Kraft, et al. (2001). COP9 signalosome-specific phosphorylation targets p53 to degradation by the ubiquitin system. Embo J 20(7): 1630-9.

Berger, M., R. Vogt Sionov, et al. (2001). A Role for the Polyproline Domain of p53 in Its Regulation by Mdm2. J Biol Chem 276(6): 3785-90.

Blaydes, J. P., M. G. Luciani, et al. (2001). Stoichiometric phosphorylation of human p53 at Ser315 stimulates p53-dependent transcription. J Biol Chem 276(7): 4699-708.

Bordoli, L., S. Husser, et al. (2001). Functional analysis of the p300 acetyltransferase domain: the PHD finger of p300 but not of CBP is dispensable for enzymatic activity. Nucleic Acids Res 29(21): 4462-71.

Bottger, V., A. Bottger, et al. (1996). Identification of novel mdm2 binding peptides by phage display. Oncogene 13(10): 2141-7.

Bottger, A., Bottger, V., Sparks, A., Liu, W. L., Howard Howard, S. F. and Lane, D. P. (1997) Design of a synthetic Mdm2-binding mini protein that activates the p53 response in vivo. Cuff Biol, 7, 860-9.

Brent, R. (2000). Genomic biology. Cell 100(1): 169-83.

Burch, L. R., Midgley, C. A., Cunie, R. A., Lane, D. P. and Hupp, T. R. (2000) Mdm2 binding to a conformationally sensitive domain on p53 can be modulated by RNA. FEBS Lett, 472, 93-98.

Buschmann, T., O. Potapova, et al. (2001). Jun NH2-terminal kinase phosphorylation of p53 on Thr-81 is important for p53 stabilization and transcriptional activities in response to stress. Mol Cell Biol 21(8): 2743-54.

Canman, C. E., D. S. Lim, et al. (1998). Activation of the ATM kinase by ionizing radiation and phosphorylation of p53. Science 281(5383): 1677-9.

Chang, N. S., N. Pratt, et al. (2001). Hyaluronidase induction of a ww domain-containing oxidoreductase that enhances tumor necrosis factor cytotoxicity. J Biol Chem 276(5): 3361-70.

Chehab, N. H., A. Malikzay, et al. (2000). Chk2/hCds1 functions as a DNA damage checkpoint in G(1) by stabilizing p53. Genes Dev 14(3): 278-88.

Chen, Y. N., Sharma, S. K., Ramsey, T. M., Jiang, L., Martin, M. S., Baker, K., Adams, P. D., Bair, K. W. and Kaelin, W. G., Jr. (1999) Selective killing of transformed cells by cyclin/cyclin-dependent kinase 2 antagonists. Proc Natl Acad Sci U S A, 96, 4325-9.

Craig, A. L., Blaydes, J. P., Burch, L. R., Thompson, A. M. and Hupp, T. R. (1999a) Dephosphorylation of p53 at Ser20 after cellular exposure to low levels of non-ionizing radiation. Oncogene, 18, 6305-12.

Craig, A. L., Burch, L., Vojtesek, B., Mikutowska, J., Thompson, A. and Hupp, T. R. (1999b) Novel phosphorylation sites of human tumour suppressor protein p53 at Ser²⁰ and Thr¹⁸ that disrupt the binding of mdm2 (mouse double minute 2) protein are modified in human cancers. Biochem J, 342, 133-141.

de Caestecker, M. P., T. Yahata, et al. (2000). The Smad4 activation domain (SAD) is a proline-rich, p300-dependent transcriptional activation domain. J Biol Chem 275(3): 2115-22.

Dornan, D. and T. R. Hupp (2001). Inhibition of p53-dependent transcription by BOX-I phospho-peptide mimetics that bind to p300. EMBO Rep 2(2): 13944.

Dumaz, N. and D. W. Meek (2000). Serine15 phosphorylation stimulates p53 transactivation, but does not directly influence interaction with HDM2. EMBO.

Dumaz, N., D. M. Milne, et al. (2001). Critical roles for the serine 20, but not the serine 15, phosphorylation site and for the polyproline domain in regulating p53 turnover. Biochem J 359(Pt 2): 459-64.

Dumaz, N. and Meek, D. W. (1999) Serine 15 phosphorylation stimulates p53 transactivation but does not directly influence interaction with HDM2. Embo J, 18, 7002-10.

Espinosa, J. M. and B. M. Emerson (2001). Transcriptional regulation by p53 through intrinsic DNA/chromatin binding and site-directed cofactor recruitment. Mol Cell 8(1): 57-69.

Friedman, P. N., X. Chen, et al. (1993). The p53 protein is an unusually shaped tetramer that binds directly to DNA [published erratum appears in Proc Natl Acad Sci USA 1993 Jun. 15;90(12):5878]. Proc Natl Acad Sci USA 90(8): 3319-23.

Gibbs, J. B. and A. Oliff (1994). Pharmaceutical research in molecular oncology. Cell 79(2): 193-8.

Goodman, R. H. and Smolik, S. (2000) CBP/p300 in cell growth, transformation, and development. Genes Dev, 14, 1553-77.

Grossman, S. R., Perez, M., Kung, A. L., Joseph, M., Mansur, C., Xiao, Z. X., Kumar, S., Howley, P. M. and Livingston, D. M. (1998) p300/MDM2 complexes participate in MDM2-mediated p53 degradation. Mol Cell, 2, 405-15.

Gu, W. and R. G. Roeder (1997). Activation of p53 sequence-specific DNA binding by acetylation of the p53 C-terminal domain. Cell 90(4): 595-606. Gu, W., X. L. Shi, et al. (1997). Synergistic activation of transcription by CBP and p53. Nature 387(6635): 819-23.

Halazonetis, T. D., L. J. Davis, et al. (1993). Wild-type p53 adopts a ‘mutant’-like conformation when bound to DNA. Embo J 12(3): 1021-8.

Hansen, S., T. R. Hupp, et al. (1996). Allosteric regulation of the thermostability and DNA binding activity of human p53 by specific interacting proteins. CRC Cell Transformation Group. J Biol Chem 271(7): 3917-24.

Hansen, S., C. A. Midgley, et al. (1996). Modification of two distinct COOH-terminal domains is required for murine p53 activation by bacterial Hsp70. J Biol Chem 271(48): 30922-8.

Haupt, Y., Maya, R., Kazaz, A. and Oren, M. (1997) Mdm2 promotes the rapid degradation of p53. Nature, 387, 296-9.

Hirao, A., Y. Y. Kong, et al. (2000). DNA damage-induced activation of p53 by the checkpoint kinase Chk2. Science 287(5459): 1824-7.

Horwitz, K. B., T. A. Jackson, et al. (1996). Nuclear receptor coactivators and corepressors. Mol Endocrinol 10(10): 1167-77.

Hupp, T. R. and D. P. Lane (1994). Allosteric activation of latent p53 tetramers. Curr Biol 4(10): 865-75.

Hupp, T. R., D. P. Lane, et al. (2000). Strategies for manipulating the p53 pathway in the treatment of human cancer. Biochem J 352 Pt 1:1-17.

Hupp, T. R., D. W. Meek, et al. (1992). Regulation of the specific DNA binding function of p53. Cell 71(5): 875-86.

Hupp, T. R., A. Sparks, et al. (1995). Small peptides activate the latent sequence-specific DNA binding function of p53. Cell 83(2): 237-45.

Jayaraman, L., E. Freulich, et al. (1997). Functional dissection of p53 tumor suppressor protein. Methods Enzymol 283: 245-56.

Keller, D. M., X. Zeng, et al. (2001). A DNA damage-induced p53 serine 392 kinase complex contains CK2, hSpt16, and SSRP1. Mol Cell 7(2): 283-92.

Kolli, S., A. M. Buchmann, et al. (2001). Antisense-mediated depletion of p300 in human cells leads to premature G1 exit and up-regulation of c-MYC. Proc Natl Acad Sci USA 98(8): 4646-51.

Kung, A. L., S. Wang, et al. (2000). Suppression of tumor growth through disruption of hypoxia-inducible transcription. Nat Med 6(12): 1335-40.

Kussie, P. H., S. Gorina, et al. (1996). Structure of the MDM2 oncoprotein bound to the p53 tumor suppressor transactivation domain [comment]. Science 274(5289): 948-53.

Lambert, P. F., Kashanchi, F., Radonovich, M. F., Shiekhattar, R. and Brady, J. N. (1998) Phosphorylation of p53 serine 15 increases interaction with CBP. J Biol Chem, 273, 33048-53.

Lau, P., P. Bailey, et al. (1999). Exogenous expression of a dominant negative RORalpha1 vector in muscle cells impairs differentiation: RORalpha1 directly interacts with p300 and myoD. Nucleic Acids Res 27(2): 411-20.

Lee, C. W., T. S. Sorensen, et al. (1998). Functional interplay between p53 and E2F through co-activator p300. Oncogene 16(21): 2695-710.

Lemon, B. and R. Tjian (2000). Orchestrated response: a symphony of transcription factors for gene control. Genes Dev 14(20): 2551-69.

Lill, N. L., S. R. Grossman, et al. (1997). Binding and modulation of p53 by p300/CBP coactivators. Nature 387(6635): 823-7.

Lin, J., J. Chen, et al. (1994). Several hydrophobic amino acids in the p53 amino-terminal domain are required for transcriptional activation, binding to mdm-2 and the adenovirus 5 E1B 55-kD protein. Genes Dev 8(10): 1235-46.

Luciani, M. G., J. R. Hutchins, et al. (2000). The C-terminal regulatory domain of p53 contains a functional docking site for cyclin A. J Mol Biol 300(3): 503-18.

Lundgren, K., R. Montes de Oca Luna, et al. (1997). Targeted expression of MDM2 uncouples S phase from mitosis and inhibits mammary gland development independent of p53. Genes Dev 11(6): 714-25.

Luo, J., A. Y. Nikolaev, et al. (2001). Negative control of p53 by Sir2alpha promotes cell survival under stress. Cell 107(2): 137-48.

Lohrum, M. A. and Vousden, K. H. (2000) Regulation and function of the p53-related proteins: same family, different rules. Trends Cell Biol, 10, 197-202.

Masumi, A. and K. Ozato (2001). Coactivator p300 Acetylates the Interferon Regulatory Factor-2 in U937 Cells following Phorbol Ester Treatment. J Biol Chem 276(24): 20973-80.

May, P., May, E. (1999) Twenty years of p53 research: structural and functional aspects of the p53 protein. Ocogene, 18, 7621-36.

Mendelsohn, A. R. and Brent, R. (1999) Protein interaction methods—toward an endgame. Science, 284, 1948-50.

Morris, L., K. E. Allen, et al. (2000). Regulation of E2F transcription by cyclin E-Cdk2 kinase mediated through p300/CBP co-activators. Nat Cell Biol 2(4): 232-9.

Muller-Tiemann, B. F., T. D. Halazonetis, et al. (1998). Identification of an additional negative regulatory region for p53 sequence-specific DNA binding. Proc Natl Acad Sci USA 95(11): 6079-84.

Murphy, M., J. Ahn, et al. (1999). Transcriptional repression by wild-type p53 utilizes histone deacetylases, mediated by interaction with mSin3a. Genes & Dev. 13(19): 2490-501. Oren, M. (1999) Regulation of the p53 tumor suppressor protein. J Biol Chem, 274, 36031-4.

Ott, M., M. Schnolzer, et al. (1999). Acetylation of the HIV-1 Tat protein by p300 is important for its transcriptional activity. Curr Biol 9(24): 1489-92.

Pelletier, G., V. Y. Stefanovsky, et al. (2000). Competitive recruitment of CBP and Rb-HDAC regulates UBF acetylation and ribosomal transcription. Mol Cell 6(5): 1059-66.

Poizat, C., V. Sartorelli, et al. (2000). Proteasome-mediated degradation of the coactivator p300 impairs cardiac transcription. Mol Cell Biol 20(23): 8643-54.

Prives, C. and J. L. Manley (2001). Why is p53 acetylated? Cell 107(7): 815-8.

Sakaguchi, K., J. E. Herrera, et al. (1998). DNA damage activates p53 through a phosphorylation-acetylation cascade. Genes Dev 12(18): 283141.

Sakaguchi, K., Saito, S., Higashimoto, Y., Roy, S., Anderson, C. W. and Appella, E. (2000) Damage-mediated phosphorylation of human p53 threonine 18 through a cascade mediated by a casein 1-like kinase. Effect on Mdm2 binding. J Biol Chem, 275, 9278-83.

Sakamuro, D., P. Sabbatini, et al. (1997). The polyproline region of p53 is required to activate apoptosis but not growth arrest. Oncogene 15(8): 887-98.

Segelmark, M., C. Barrett, et al. (2000). Expression of p300-truncated fragments results in the modulation of apoptosis in rat mesangial cells. Kidney Int 57(5): 1873-81.

Shieh, S. Y., Ahn, J., Tamai, K., Taya, Y. and Prives, C. (2000) The human homologs of checkpoint kinases Chk1 and Cds1 (Chk2) phosphorylate p53 at multiple DNA damage-inducible sites. Genes Dev, 14, 289-300.

Shieh, S. Y., Ikeda, M., Taya, Y. and Prives, C. (1997) DNA damage-induced phosphorylation of p53 alleviates inhibition by MDM2. Cell, 91, 325-34.

Shikama, N., Lee, C. W., France, S., Delavaine, L., Lyon, J., Krstic-Demonacos, M. and La Thangue, N. B. (1999) A novel cofactor for p300 that regulates the p53 response. Mol Cell, 4, 365-76.

Snowden, A W., L. A. Anderson, et al. (2000). A novel transcriptional repression domain mediates p21 (WAF1/CIP1) induction of p300 transactivation [published erratum appears in Mol Cell Biol 2000 Jul;20(14):5360]. Mol Cell Biol 20(8): 2676-86.

Sterner, D. E. and S. L. Berger (2000). Acetylation of histones and transcription-related factors. Microbiol Mol Biol Rev 64(2): 435-59.

Thompson, P. R., H. Kurooka, et al. (2001). Transcriptional coactivator protein p300. Kinetic characterization of its histone acetyltransferase activity. J Biol Chem 276(36): 33721-9.

Unger, T., Juven-Gershon, T., Moallem, E., Berger, M., Vogt Sionov, R., Lozano, G., Oren, M. and Haupt, Y. (1999) Critical role for ser20 of human p53 in the negative regulation of p53 by mdm2. Embo J, 18, 1805-14.

Van Orden, K., H. A. Giebler, et al. (1999). Binding of p53 to the KIX domain of CREB binding protein. A potential link to human T-cell leukemia virus, type I-associated leukemogenesis. J Biol Chem 274(37): 26321-8.

Venot, C., M. Maratrat, et al. (1998). The requirement for the p53 proline-rich functional domain for mediation of apoptosis is correlated with specific PIG3 gene transactivation and with transcriptional repression. EMBO J. 17(16): 4668-4679.

Vo, N. and R. H. Goodman (2001). CREB-binding protein and p300 in transcriptional regulation. J Biol Chem 276(17): 13505-8.

Vogelstein, B., D. Lane, et al. (2000). Surfing the p53 network. Nature 408(6810): 307-10.

Walker, K. K. and A. J. Levine (1996). Identification of a novel p53 functional domain that is necessary for efficient growth suppression. Proc Natl Acad Sci USA 93(26): 15335-40.

Wang, L., Q. Wu, et al. (2001). Analyses of p53 target genes in the human genome by bioinformatic and microarray approaches. J Biol Chem 276(47): 43604-10.

Wang, Y. and C. Prives (1995). Increased and altered DNA binding of human p53 by S and G2/M but not G1 cyclin-dependent kinases. Nature 376(6535): 88-91.

Webley, K., Bond, J. A., Jones, C. J., Blaydes, J. P., Craig, A., Hupp, T. and Wynford-Thomas, D. (2000) Posttranslational modifications of p53 in replicative senescence overlapping but distinct from those induced by DNA damage. Mol Cell Biol, 20, 2803-8.

Webster, G. A. and N. D. Perkins (1999). Transcriptional cross talk between NF-kappaB and p53. Mol Cell Biol 19(5): 3485-95.

Yuan, Z. M., Y. Huang, et al. (1999). Function for p300 and not CBP in the apoptotic response to DNA damage. Oncogene 18(41): 5714-7.

Yuan, Z. M., Y. Huang, et al. (1999). Role for p300 in stabilization of p53 in the response to DNA damage. J Biol Chem 274(4): 1883-6.

Zhang, W. and J. J. Bieker (1998). Acetylation and modulation of erythroid Kruppel-like factor (EKLF) activity by interaction with histone acetyltransferases. Proc Natl Acad Sci USA 95(17): 9855-60.

Zhu, J., J. Jiang, et al. (1999). Differential regulation of cellular target genes by p53 devoid of the PXXP motifs with impaired apoptotic activity. Oncogene 18(12): 2149-55.

Zilfou, J. T., W. H. Hoffman, et al. (2001). The Corepressor mSin3a Interacts with the Proline-Rich Domain of p53 and Protects p53 from Proteasome-Mediated Degradation. Mol Cell Biol 21(12): 3974-85.

1. A method for identifying a substance capable of modulating an interaction between (i) a p53 polypeptide or a homologue thereof, or a derivative thereof, and (ii) a p300 polypeptide, or a homologue thereof, or a derivative thereof, which method comprises:
a) providing a p53 polypeptide or a homologue, or a derivative thereof, as a first component;
b) providing a p300 polypeptide or a homologue, or a derivative thereof, as a second component;
c) contacting the two components with a test substance under conditions that would permit the two components to bind in the absence of said test substance; and
d) determining whether said substance modulates the interaction between the first and second components.

2. The method according to claim 1 further comprising the steps of
e) administering a substance which has been determined to disrupt the interaction between the first and second components to an animal cell; and
f) determining the effect of the substance on the cell.

3. The method according to either of claims 1 or 2 wherein said derivative of p53 comprises at least a region having substantial homology to the BOX-I domain of p53.

4. The method according to claim 3 wherein Ser¹⁵, Thr¹⁸ and/or Ser²⁰ of the BOX-I domain is phosphorylated.

5. The method according to claim 3 wherein Ser²⁰ of the BOX-I domain is phosphorylated.

6. The method according to claim 3 wherein Ser¹⁵, Thr¹⁸ and/or Ser²⁰ are independently substituted by an aspartate or glutamate residue.

7. The method according to either of claims 1 or 2 wherein said derivative of p53 comprises at least a polyproline region of p53 having the sequence PRMPEAAPPVAPAPAAPTPAAPAPAPSWP.

8. The method according to claim 5 wherein said derivative of p300 comprises at least a region which binds to said p53 derivative comprising phosphorylated Ser²⁰, said region comprising at least a portion of the sequence
CASSRQIISHWKNCTRHDCPVCLPLKNAGDKRNQQPILTGAPVGLGNPSSLGVGQQSAPNL
STVSQIDPSSIERAYAALGLPYQVNQMPTQPQVQAKNQQNQQPGQSPQGMRPMSNMSASP
MGVNGGVGVQTPSLLSDSMLHSAINSQNPMMSENASVPSLGPMPTAAQPSTTG; and/or
AAGQVTPPTPPQTAQPPLPGPPPTAVEMAMQIQRAAETQRQMAHVQIFQRPIQHQMPPMTP
MAPMGMNPPPMTRGPSGHLEPGMGPTGMQQQPPWSQGGLPQPQQLQSGMPRPAMMSV
AQHGQPLNMAPQPGLGQVGISPLKPGTVSQQALQNLLRTLRSPSSPLQQQQVLSILHANPQL
LAAFIKQRAAKYANSNPQPIPGQPGMPQGQPGLQPPTMPGQQGVHSNPAMQNMNPMQAG
VQRAGLPQQQPQQQLQPPMGGMSPQAQQMNMNHNTMPSQFRDILRRQQMMQQQQQQG
AGPGIGPGMANHNQFQQPQGVGYPPQPQQRMQHHMQQMQQGNMGQIGQLPQALGAEA
GASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNS
MLSQLASNPGMANLHGASATDLGLSTDNSDLNSNLSQSTLDIH.

9. The method according to any one of claims 1, 2 or 7 wherein said derivative of p300 comprises at least a region which binds to a polyproline region of p53, said region comprising at least a portion of the sequence
PAMGMNTGTNAGMNPGMLAAGNGQGIMPNQVMNGSIGAGRGRQDMQYPNPGMGSAGNLLT
EPLQQGSPQMGGQTGLRGPQPLKMGMMNNPNPYGSPYTQNPGQQIGASGLGLQIQTKTVLSN
NLSPFAMDKKAVPGGGMPNMGQQPAPQVQQPGLVTPVAQGMGSGAHTADPEKAENVVEPGP
PSAKRPKLSSPALSASASDGTDFGSLFDLEHDLP; and/or
TCNECKHHVETRWHCTVCEDYDLCITCYNTKNHDHKMEKLGLGLDDESNNQQAAATQSPGDSR
RLSIQRCIQSLVHACQCRNANCSLPSCQKMKRVVQHTKGCKRKTNGGCPICKQLIALCCYHAKH
CQENKCPVPFCLNIKQKLRQQQLQHRLQQAQMLRRRMASMQRTGVVGQQQGLPSPTPATPTT
PTGQQPTTPQTPQPTSQPQPTPPNSMPPYLPRTQAAGPVSQGKAAGQVPPTPPQTAQPPLPG
PPPTAVEMAMQIQRAAETQRQMAHVQIFQRPIQHQMPP.

10. A substance capable of modulating an interaction between (i) a p53 polypeptide or a homologue thereof, or a derivative thereof, and (ii) p300 or a homologue thereof, or a derivative thereof, identified by the method according to any preceding claim for use in treating the human or animal body by therapy or for use in diagnosis, whether or not practised on the human or animal body.

11. A substance capable of modulating an interaction between (i) a p53 polypeptide or a homologue thereof, or a derivative thereof, and (ii) p300 or homologues thereof, or derivatives thereof, identified by the method according to any one of claims 1 to 9 for use in regulating the cell cycle of a mammalian cell.

12. A method of regulating the cell cycle in a mammalian cell, which method comprises administering to said cell a substance capable of modulating an interaction between (i) a p53 polypeptide or a homologue thereof, or a derivative thereof, and (ii) p300 or a homologue thereof, or a derivative thereof.

13. A peptide comprising the sequence
YXXWXLL
where Y is S^{PO3} or D and X represents any amino acid.

14. A peptide according to claim 13 comprising the sequence
EPPLSQETFDDLWKLLPEN
for use in modulating the binding of p53 to p300.

15. A peptide comprising the sequence
DEAPRMPEAAPPVAPAPAAPTPAAPAPAPSWPL
for use in modulating the binding of p53 to p300.

16. Use of a peptide according to any of claims 13 to 15 for modulating the binding of p53 to p300.

17. Use of a peptide comprising the sequence PXnP wherein x represents any amino acid and n is 1 to 3 for modulating the binding of p53 to p300.
